SEQUENCE LISTING (24 sequences)

SEQ ID NO: 1 - protein, 785 aa, synthetic construct
  REGION 1..785: note = synthetic construct
SQIEVKDVTDTTALITWSDDFGEYVWCELTYGIKDVPGDRTTIDLWYHHAHYSIGNLKPDTEYEVSLICRSGDMSSNPAKETFTTGGGGGGGGGGGGGGGRLDAPSQIEVKDVTDTTALITWSDDFGEYVWCELTYGIKDVPGDRTTIDLWYHHAHYSIGNLKPDTEYEVSLICRSGDMSSNPAKETFTTGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 83 aa, synthetic construct
  REGION 1..83: note = synthetic construct
IEVKDVTDTTALITWSDDFGEYVWCELTYGIKDVPGDRTTIDLWYHHAHYSIGNLKPDTEYEVSLICRRGDMSSNPAKETFTT

SEQ ID NO: 3 - protein, 83 aa, synthetic construct
  REGION 1..83: note = synthetic construct
IEVKDVTDTTALITWSDDFGEYVWCELTYGIKDVPGDRTTIDLWYHHAHYSIGNLKPDTEYEVSLICRSGDMSSNPAKETFTT

SEQ ID NO: 4 - protein, 585 aa, synthetic construct
  REGION 1..585: note = synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - skipped

SEQ ID NO: 6 - protein, 5 aa, synthetic construct
  REGION 1..5: note = synthetic construct
ALITW

SEQ ID NO: 7 - protein, 10 aa, synthetic construct
  REGION 1..10: note = linker
GGGGSGGGGS

SEQ ID NO: 8 - protein, 15 aa, synthetic construct
  REGION 1..15: note = linker
GGGGSGGGGSGGGGS

SEQ ID NO: 9 - protein, 10 aa, synthetic construct
  REGION 1..10: note = linker
GGGGGGGGGG

SEQ ID NO: 10 - protein, 15 aa, synthetic construct
  REGION 1..15: note = linker
GGGGGGGGGGGGGGG

SEQ ID NO: 11 - protein, 7 aa, synthetic construct
  REGION 1..7: note = synthetic construct
KDVTDTT

SEQ ID NO: 12 - protein, 9 aa, synthetic construct
  REGION 1..9: note = synthetic construct
SDDFGEYVW

SEQ ID NO: 13 - protein, 7 aa, synthetic construct
  REGION 1..7: note = synthetic construct
KDVPGDR

SEQ ID NO: 14 - protein, 6 aa, synthetic construct
  REGION 1..6: note = synthetic construct
WYHHAH

SEQ ID NO: 15 - protein, 8 aa, synthetic construct
  REGION 1..8: note = synthetic construct
GNLKPDTE

SEQ ID NO: 16 - protein, 10 aa, synthetic construct
  REGION 1..10: note = synthetic construct
RSGDMSSNPA

SEQ ID NO: 17 - protein, 7 aa, synthetic construct
  REGION 1..7: note = synthetic construct
CELTYGI

SEQ ID NO: 18 - protein, 5 aa, synthetic construct
  REGION 1..5: note = synthetic construct
TTIDL

SEQ ID NO: 19 - skipped

SEQ ID NO: 20 - protein, 7 aa, synthetic construct
  REGION 1..7: note = synthetic construct
YEVSLIC

SEQ ID NO: 21 - protein, 6 aa, synthetic construct
  REGION 1..6: note = synthetic construct
KETFTT

SEQ ID NO: 22 - protein, 85 aa, synthetic construct
  REGION 1..85: note = synthetic construct
SQIEVKDVTDTTALITWSDDFGEYVWCELTYGIKDVPGDRTTIDLWYHHAHYSIGNLKPDTEYEVSLICRSGDMSSNPAKETFTT

SEQ ID NO: 23 - protein, 10 aa, synthetic construct
  REGION 1..10: note = synthetic construct
RLDAPSQIEV

SEQ ID NO: 24 - protein, 5 aa, synthetic construct
  REGION 1..5: note = synthetic construct
SQIEV

(19) United States
(12) Patent Application Publication
(10) Pub. No.: US 2024/0084024 A1
(43) Pub. Date: Mar. 14, 2024

(54) CD40L ANTAGONIST AND USES THEREOF IN THE TREATMENT OF LUPUS NEPHRITIS

(71) Applicant: Viela Bio, Inc., Deerfield, IL (US)

(72) Inventors: Gabor G. ILLEI, Rockville, MD (US); Ilias ALEVIZOS, Edgewater, NJ (US); Jorn DRAPPA, La Jolla, CA (US); William A. REES, North Bethesda, MD (US)

(21) Appl. No.: 18/509,836

(22) Filed: Nov. 15, 2023

Related U.S. Application Data
(63) Continuation of application No. PCT/US2022/030290, filed on May 20, 2022.
(60) Provisional application No. 63/191,514, filed on May 21, 2021.
(60) Provisional application No. 63/235,520, filed on Aug. 20, 2021.

(51) Int. Cl. C07K 16/28; A61K 31/573; A61K 31/664; A61K 47/10; A61P 13/12

(52) U.S. Cl. CPC C07K 16/2875; A61K 31/573; A61K 31/664; A61K 47/10; A61P 13/12; A61K 2039/545

(57) ABSTRACT

A human CD40L-specific Tn3 molecule and therapeutic uses thereof for the treatment of lupus nephritis are provided herein. Also provided are therapeutic regimens that comprise a CD40L-specific Tn3 scaffold.

### CROSS REFERENCE TO RELATED APPLICATIONS

This application is a continuation of International Application No. PCT/US2022/030290, filed May 20, 2022, which claims priority to U.S. Provisional Patent Application No. 63/191,514 filed May 21, 2021, and U.S. Provisional Patent Application No. 63/235,520 filed Aug. 20, 2021, both incorporated by reference herein in their entirety for all purposes.

### REFERENCE TO AN ELECTRONIC SEQUENCE LISTING

The contents of the electronic sequence listing (HOPA_013_02US_SeqList_ST26.xml; Size: 30,067 bytes; and Date of Creation: Oct. 17, 2023) are herein incorporated by reference in its entirety.

### TECHNICAL FIELD

The present disclosure is related to CD40L antagonists and methods of making and utilizing the same for the prevention and treatment of lupus nephritis.

### BACKGROUND

The CD40/CD40L pathway plays a critical role in driving humoral immune responses and has been implicated in the pathogenesis of several autoimmune diseases. CD40 is constitutively expressed on a variety of antigen presenting cells, including dendritic cells (DCs), macrophages, and B cells (S. Sugio, A et al, 1999), and can also be expressed on non-hematopoietic cells.

Expression of the CD40 ligand, CD40L (also known as CD154), is highly regulated and is mostly found on activated CD4+ T cells (Lederman et al, 1992). CD40/CD40L interactions between B cells and activated T cells are essential for mounting effective humoral responses to T-dependent antigens (M. Croft et al., 1994; T. M. Foy et al., 1994; J. B. Splawski et al., 1994). The CD40/CD40L axis drives B cell expansion, differentiation and isotype switching in vitro (R. J. Armitage et al., 1992; P. Garside et al., 1998; D. Hollenbaugh et al., 1992; R. J. Noelle et al., 1992). In vivo, CD40 signaling is required for germinal center (GC) formation, somatic hyper mutation and the generation of memory B cells and long-lived plasma cells (T. M. Foy et al., 1994; T. M. Foy et al., 1993; S. Han et al., 1995; T. Kawabe et al., 1994). CD40 or CD40L defects in humans lead to X-linked hyperimmunoglobulin syndrome, a disease characterized by impaired isotype class switching, which manifests as high levels of serum IgM with low to no detectable IgG, IgA or IgE and increased susceptibility to infections (R. C. Allen et al., 1993; A. Aruffo et al., 1993; J. P. DiSanto et al., 1993).

Fusion of a bivalent CD40L-specific Tn3 protein to human serum albumin (HSA) resulted in a molecule, i.e., VIB4920, that was able to bind human CD40L and prevent its interaction with CD40 receptor. Consistent with this disruption in CD40L/CD40 interaction, VIB4920 was able to potently inhibit activation and differentiation of human B cells in vitro by blocking CD40 signaling events.

Currently available methods are directed towards treating autoimmune diseases and not towards preventing such diseases. Further, conventional treatment options for autoimmune diseases include immunosuppressant drugs that are associated with a wide range of side effects. Thus, there is a need for better therapeutic alternatives for treating and preventing autoimmune diseases, in particular lupus nephritis. The present disclosure addresses these needs.

### BRIEF SUMMARY

The description provides for a method for treating lupus nephritis in a subject in need thereof comprising: administering a Tn3 scaffold comprising a CD40L-specific monomer subunit to the subject; wherein the Tn3 scaffold binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 weeks for at least 2 doses, and is administered about once a month thereafter.

The description provides for a method for treating lupus nephritis in a subject in need thereof comprising: administering a Tn3 scaffold comprising a CD40L-specific monomer subunit to the subject; wherein the Tn3 scaffold binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 weeks for at least 3 doses, and is administered about once a month thereafter.

The description provides for a method for treating lupus nephritis in a subject in need thereof comprising: administering a Tn3 scaffold comprising a CD40L-specific monomer subunit in combination with mycophenolate mofetil (MMF) to the subject; wherein the Tn3 scaffold binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 1500 mg.

The description provides methods for treating lupus nephritis in a subject in need thereof comprising: administering a Tn3 scaffold comprising a CD40L-specific monomer subunit in combination with mycophenolate mofetil (MMF) and methylprednisolone to the subject; wherein the Tn3 scaffold binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 1500 mg.

The description provides for a method for treating lupus nephritis in a subject in need thereof comprising: administering a Tn3 scaffold comprising a CD40L-specific monomer subunit in combination with cyclophosphamide to the subject, wherein the Tn3 scaffold binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold is administered at a dose of about 1500 mg. In aspects, cyclophosphamide is administered for at least about eight, ten, twelve or more weeks prior to the first administration of the Tn3 scaffold. In aspects, the cyclophosphamide is administered for at least about eight weeks prior to the first administration of the Tn3 scaffold. In aspects, the cyclophosphamide is administered for at least about ten weeks prior to the first administration of the Tn3 scaffold. In aspects, the cyclophosphamide is administered for at least about twelve weeks prior to the first administration of the Tn3 scaffold.

In aspects, the Tn3 scaffold is administered once about every two, three, four weeks, or about once a month. In aspects, the Tn3 scaffold is administered once about every 2 weeks for at least 2 doses, and is administered about once a month thereafter. In aspects, the Tn3 scaffold is administered once about every 2 weeks for at least 3 doses, and is administered about once a month thereafter.

In aspects, the Tn3 scaffold is administered intravenously.

In aspects, the Tn3 scaffold comprises two CD40L-specific monomer subunits connected in tandem. In aspects, the Tn3 scaffold binds CD40L and prevents binding of CD40L to CD40 and/or disrupts CD40 mediated signaling. In aspects, the Tn3 scaffold (a) binds CD40L thereby reducing or preventing binding of CD40L to CD40; (b) reduces or eliminates CD40 mediated signaling; or (c) a and b. In aspects, the at least one CD40L-specific monomer subunit is fused or conjugated to a heterologous moiety selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, and a cytokine. In aspects, at least one CD40L-specific monomer subunit is conjugated to PEG or is fused to a human serum albumin (HSA). In aspects, said HSA is a variant HSA having the amino acid sequence of SEQ ID NO: 4. In aspects, the Tn3 scaffold comprises the sequence of SEQ ID NO: 1. In aspects, the Tn3 scaffold is VIB4920.

In aspects, the subject is further administered prednisone. In aspects, the subject received one or more standard of care therapies prior to the administration of the Tn3 scaffold. In aspects, the standard of care therapy is MMF. In aspects, the standard of care therapy is methylprednisolone. In aspects, the standard of care therapy is cyclophosphamide. In aspects, the standard of care therapy is prednisone.

Provided are methods of treating lupus nephritis comprising administering to a subject in need thereof; a) a preparative regime that comprises administration of at least one immunosuppressant in an amount sufficient to reduce an immune response in the subject; and b) about 1000-2000 mg of Dazodalibep, wherein the Dazodalibep is administered once about every 2 weeks for at least 2 doses, and is administered about once a month thereafter.

Provided are methods of treating lupus nephritis comprising administering to a subject in need thereof: a) a preparative regime that comprises: i. administration of an immunosuppressant in an amount sufficient to reduce an immune response in the subject; and ii. administration of a corticosteroid in an amount sufficient to reduce inflammation in the student; and b) about 1000-2000 mg of Dazodalibep, wherein the Dazodalibep is administered once about every 2 weeks for at least 2 doses, and is administered about once a month thereafter.

In aspects, an immunosuppressant is selected from the group consisting of: Azathioprine, Mycophenolate mofetil, Cyclosporine, Methotrexate, Leflunomide, Cyclophosphamide, Chlorambucil, Nitrogen mustard, and combinations thereof. In aspects, an immunosuppressant is selected from the group consisting of: azathioprine, mycophenolate mofetil, cyclophosphamide, and cyclosporine. In aspects, an immunosuppressant is mycophenolate mofetil. In aspects, an immunosuppressant is cyclophosphamide. In aspects, an immunosuppressant is administered for at least about eight, nine, ten, eleven, twelve or more weeks prior to a first administration of the Dazodalibep. In aspects, cyclophosphamide is administered every 4 weeks. In aspects, cyclophosphamide is administered every 2 weeks. In aspects, about 1200-1800 mg of the Dazodalibep is administered. In aspects, about 1500 mg of Dazodalibep is administered. In aspects, a preparative regimen further comprises administering a corticosteroid to the subject. In aspects, a corticosteroid is prednisone. In aspects, administering of the corticosteroid is tapered.

In aspects, in any of the preceding methods, a Tn3 scaffold monomer comprises a beta A strand that comprises SEQ ID NO: 5, SEQ ID NO: 23, or SEQ ID NO: 24, a beta B strand that comprises SEQ ID NO: 6, a beta C strand that comprises SEQ ID NO: 17, a beta D strand that comprises SEQ ID NO: 18, a beta E strand that comprises SEQ ID NO: 19, a beta F strand that comprises SEQ ID NO: 20, and a beta G strand that comprises SEQ ID NO: 21. In aspects, the beta A strand consists of SEQ ID NO: 5. In aspects, the beta A strand consists of SEQ ID NO: 23. In aspects, the beta A strand consists of SEQ ID NO: 24. In aspects, the beta B strand consists of SEQ ID NO: 6. In aspects, the beta C strand consists of SEQ ID NO: 17. In aspects, the beta D strand consists of SEQ ID NO: 18. In aspects, the beta E strand consists of SEQ ID NO: 19. In aspects, the beta F strand consists of SEQ ID NO: 20. In aspects, the beta G strand consists of SEQ ID NO: 21.

### DETAILED DESCRIPTION

Provided herein are Tn3 scaffolds that are anti-cluster of differentiation (CD) 40 ligand (CD40L)-third fibronectin type III (Fn3) protein domain of human Tenascin C (Tn3) protein fusion proteins and methods of using the same in autoimmune disease. In aspects, compositions and methods provided are utilized for the treatment of lupus nephritis.

Also provided are methods comprising administering Tn3 scaffolds with immunosuppressants and/or corticosteroids for the treatment of lupus nephritis.

### Definitions

Unless defined otherwise, all technical and scientific terms used herein have the same meaning as commonly understood by one of ordinary skill in the art to which the subject matter pertains. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used in this specification and the appended claims, the singular forms “a,” “an” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5. The term “about” also accounts for typical error or imprecision in measurement of values.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. The terms “subject” and “patient” are used interchangeably herein. In aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, or the like.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, the term “normal subject” refers to any healthy subject, e.g., a human or a non-human mammal, not affected with any disease or suspected of being affected with a disease or condition.

As used herein, “treating” or “treat” describes the management and care of a subject for the purpose of combating a disease, condition, or disorder and includes the administration of VIB4920 used in the methods described herein to alleviate the symptoms or complications of a disease, condition or disorder, or to eliminate the disease, condition or disorder. Thus, the term “treat” or “treating” refers to both therapeutic measures and prophylactic or preventative measures, wherein the objective is to prevent, slow down (lessen), or ameliorate the progression of a disease (e.g., lupus nephritis). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total). The term “treat” can also include treatment of a cell in vitro or an animal model.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 is a study flow diagram summarizing the trial design provided herein. In brief, up to 114 eligible subjects with active LN will receive induction therapy with MMF and methylprednisolone beginning at Week 0. Subjects will receive a total of 1000 mg of methylprednisolone according to either of the following schedules: 1000 mg methylprednisolone IV at Day 0, or 500 mg methylprednisolone IV at Day 0 and at Day 1. Subjects will also receive prednisone 25 mg per day beginning at Day 0, or on the day after completion of methylprednisolone and tapered to 5 mg per day at Week 8. Subjects will be assessed at Week 8 for a renal response. Sixty-six subjects with a urine protein-to-creatinine ratio (UPCR) >0.75 will be randomized 2:1 to VIB4920 versus placebo at Week 10. Subjects who are not eligible for randomization will complete study participation after the Week 8 study visit, and further care will be provided according to the judgment of the site investigator or treating physician. Randomized subjects will receive VIB4920 1500 mg or placebo intravenously at Weeks 10, 12, 14, 18, 22, 26, 30, and 34, and will continue MMF 2-3 g per day and prednisone 5 mg per day. The primary endpoint will be assessed at Week 38, and subjects followed until Week 60.

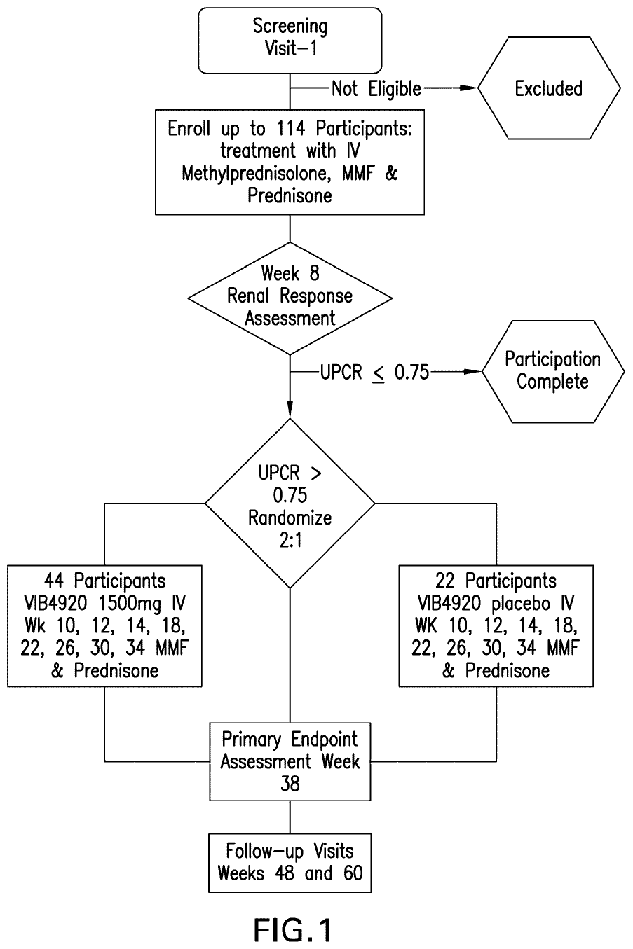

Described herein are methods for treating an autoimmune disorder using a Tn3 scaffold comprising a CD40L-specific monomer subunit. In aspects, the Tn3 scaffold is used in methods of treating lupus. In aspects, the lupus is SLE. In one aspect, the lupus is lupus nephritis (LN). In aspects, the methods comprise treating lupus nephritis in a subject in need thereof by administering the Tn3 scaffold in combination with mycophenolate mofetil (MMF). In aspects, the methods comprise treating lupus nephritis in a subject in need thereof by administering the Tn3 scaffold in combination with MMF and methylprednisolone. In aspects, the methods comprise treating lupus nephritis in a subject in need thereof by administering the Tn3 scaffold in combination with cyclophosphamide. In aspects, the Tn3 scaffold binds to CD40L. In aspects, the monomer subunit of the Tn3 scaffold comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16. In a particular aspect, the Tn3 scaffold is VIB4920. In aspects, provided are methods to reduce or eliminate binding of CD40L in active LN. In aspects, provided are also methods to reduce CD40L binding in active LN as determined by any one of: (1) achieving a renal response: reducing or eliminating anti-dsDNA antibodies; (3) reducing or eliminating hypocomplementemia; (4) reducing or eliminating SLE disease activity and/or damage accrual; and/or (5) preventing renal failure.

Tn 3 Scaffolds

Provided herein are compositions that comprise a Tn3 scaffold for use in treating lupus nephritis. In aspects, the compositions may comprise the amino acid sequences as described in Int'l Appl. Nos. PCT/US2012/059477 and PCT/US2019/052997, which are incorporated herein by reference in their entireties. In aspects, the compositions may comprise or consist of the amino acid sequence as shown in SEQ ID NO: 1 (referred to herein as VIB4920 or Dazodalibep, used interchangeably herein). VIB4920 comprises a bivalent CD40L-specific Tn3 protein fused to a human serum albumin (HSA) protein.

In aspects, the Tn3 scaffold comprising a CD40L-specific monomer subunit, wherein the monomer subunit comprises seven beta strands designated A, B, C, D, E, F, and G, and six loop regions designated AB, BC, CD, DE, EF, and FG, and wherein the Tn3 scaffold specifically binds to CD40L. In aspects, the Tn3 scaffold comprises a single CD40L-specific monomer subunit. In aspects, the Tn3 scaffold comprises two CD40L-specific monomer subunits connected in tandem. In aspects, the Tn3 scaffold comprises two CD40L-specific monomer subunits which are directly connected. In aspects, two CD40L-specific monomer subunits are connected by a linker. In other aspects, the linker comprises a peptide linker, which can be a flexible peptide linker. In aspects, the peptide linker comprises a (GₘX)ₙ sequence wherein X is Serine (S), Alanine (A), Glycine (G), Leu (L), Isoleucine (I), or Valine (V); m and n are integer values; m is 1, 2, 3 or 4; and, n is 1, 2, 3, 4, 5, 6, or 7.

In aspects, the Tn3 scaffold comprises a linker which comprises a functional moiety. In aspects, this functional moiety is an immunoglobulin or a fragment thereof. In aspects, this immunoglobulin or fragment thereof comprises an Fc domain. In aspects, this Fc domain fails to induce at least one FcγR-mediated effector function. In aspects, this at least one FcγR-mediated effector function is ADCC.

In aspects, the Tn3 scaffold comprises seven beta strands designated A, B, C, D, E, F, and G, and six loop regions designated AB, BC, CD, DE, EF, and FG, wherein the AB loop comprises or consists of SEQ ID NO: 11, the BC loop comprises or consists of SEQ ID NO: 12, the CD loop comprises or consists of SEQ ID NO: 13, the DE loop comprises or consists of SEQ ID NO: 14, the EF loop comprises or consists of SEQ ID NO: 15, and the FG loop comprises or consists of SEQ ID NO: 16. In aspects, the Tn3 scaffold comprises or consists of SEQ ID NO: 1. In aspects, beta strand A comprises or consists of SEQ ID NO: 5, beta strand B comprises or consists of SEQ ID NO: 6, beta strand C comprises or consists of SEQ ID NO: 17, beta strand D comprises or consists of SEQ ID NO: 18, beta strand E comprises or consists of SEQ ID NO: 19, beta strand F comprises or consists of SEQ ID NO: 20, and beta strand G comprises or consists of SEQ ID NO: 21.

In aspects, one or more CD40L-specific Tn3 monomers have a beta strand A comprising or consisting of IEV (SEQ ID NO: 5), RLDAPSQIEV (SEQ ID NO: 23), or SQIEV (SEQ ID NO: 24). In aspects, a Tn3 scaffold may comprise one or more CD40L-specific Tn3 monomers having the same or different beta strand A sequences. For example, a first CD40L-specific Tn3 monomer beta strand A may comprise or consist of IEV (SEQ ID NO: 5) and a second CD40L-specific Tn3 monomer beta strand A may comprise or consist of RLDAPSQIEV (SEQ ID NO: 23) or SQIEV (SEQ ID NO: 24).

In aspects, a Tn3 scaffold monomer of the disclosure comprises a beta A strand that comprises SEQ ID NO: 5, SEQ ID NO: 23, or SEQ ID NO: 24, a beta B strand that comprises SEQ ID NO: 6, a beta C strand that comprises SEQ ID NO: 17, a beta D strand that comprises SEQ ID NO: 18, a beta E strand that comprises SEQ ID NO: 19, a beta F strand that comprises SEQ ID NO: 20, and a beta G strand that comprises SEQ ID NO: 21. In aspects, the beta A strand consists of SEQ ID NO: 5. In aspects, the beta A strand consists of SEQ ID NO: 23. In aspects, the beta A strand consists of SEQ ID NO: 24. In aspects, the beta B strand consists of SEQ ID NO: 6. In aspects, the beta C strand consists of SEQ ID NO: 17. In aspects, the beta D strand consists of SEQ ID NO: 18. In aspects, the beta E strand consists of SEQ ID NO: 19. In aspects, the beta F strand consists of SEQ ID NO: 20. In aspects, the beta G strand consists of SEQ ID NO: 21.

The Tn3 scaffold may have the amino acid sequence as shown in SEQ ID NO: 1 and described above or it may have one or more amino acid residues changes relative to the amino acid sequence as shown in SEQ ID NO: 1. For example, if the Tn3 scaffold has amino acid sequence changes relative to those shown in SEQ ID NO: 1, the changes may be to one of the linkers. The Tn3 scaffold comprises a Gly15 linker separating two CD40L-specific monomers and a Gly 10 linker separating a CD40L-specific monomer from an HSA sequence. Both or one of these linkers may be altered, and may be replaced with an amino acid sequence of (GₘX)ₙ wherein X is Serine (S), Alanine (A), Glycine (G), Leu (L), Isoleucine (I), or Valine (V); m and n are integer values; m is 1, 2, 3 or 4; and, n is 1, 2, 3, 4, 5, 6, or 7. For example, one or both linkers may be altered to have an amino acid sequence that comprises one of GGGGSGGGGS (SEQ ID NO: 7), GGGGSGGGGSGGGGS (SEQ ID NO: 8), GGGGGGGGGG (SEQ ID NO: 9) or GGGGGGGGGGGGGGG (SEQ ID NO: 10). If the Tn3 scaffold has an amino acid sequence relative to the amino acid sequence as provided in SEQ ID NO: 1, it may be due to a changes or changes in the HSA amino acid sequence fused to the two CD40L-specific monomers. The HSA fused to the two CD40L-specific monomers may be altered to relative to the HSA fused to the two CD40L-specific Tn3 monomers, except for at least one amino acid substitution, numbered relative to the position in full length mature HSA, at a position selected from the group consisting of 407, 415, 463, 500, 506, 508, 509, 511, 512, 515, 516, 521, 523, 524, 526, 535, 550, 557, 573, 574, and 580; wherein the at least one amino acid substitution does not comprise a lysine (K) to glutamic acid (E) at position 573.

An exemplary Tn3 scaffold is shown in Table 1. In aspects, a Tn3 scaffold comprises at least about or at most about 50%, 55%, 60%, 65%, 70%, 75%, 80%, 85%, 90%, 95%, 96%, 97%, 98%, 99%, or up to about 100% identity with any one of SEQ ID NO: 1-SEQ ID NO: 24 shown in Table 1. In aspects, any one of the sequences from Table 1 can be modified. In aspects, a modification comprises one or more truncations, deletions, insertions, and combinations thereof. A modification can occur at any of the residues provided in Table 1 and in any number of residues from Table 1. In aspects, a modification can comprise from 0-3, 0-5, 0-10, 0-20, 1-3, 1-5, 1-10, 1-20, 3-8, 3-10, 3-15, 5-8, 5-10, or 5-20 residues. In aspects, a modification can occur in 0, 1, 2, 3, 4, 5, 6, 7, 8, 9, 10, 11, 12, 13, 14, 15, 20, 30, 40, 50, 60, 70, 80, 90, 100, 200, 300, 400, or 450 residues.

TABLE 1
Exemplary sequences for a Tn3 scaffold comprising a CD40L-specific monomer
subunit

| SEQ | | |
|---|---|---|
| ID NO | ID | Sequence |
| 1 | Dazodalibep | SQIEVKDVTDTTALITWSDDFGEYVWCELTYGIKDVPGDRT |
| | “VIB4920” | TIDLWYHHAHYSIGNLKPDTEYEVSLICRSGDMSSNPAKETF |
| | (342-G15-342- | TTGGGGGGGGGGGGGGGRLDAPSQIEVKDVTDTTALITWS |
| | G10- | DDFGEYVWCELTYGIKDVPGDRTTIDLWYHHAHYSIGNLK |
| | HSAC34S | PDTEYEVSLICRSGDMSSNPAKETFTTGGGGGGGGGGDAH |
| | Bivalent | KSEVAHRFKDLGEENFKALVLIAFAQYLQQSPFEDHVKLVN |
| | construct 2- | EVTEFAKTCVADESAENCDKSLHTLFGDKLCTVATLRETYG |
| | All GLY | EMADCCAKQEPERNECFLQHKDDNPNLPRLVRPEVDVMCT |
| | linkers HSA | AFHDNEETFLKKYLYEIARRHPYFYAPELLFFAKRYKAAFT |
| | underlined) | ECCQAADKAACLLPKLDELRDEGKASSAKQRLKCASLQKF |
| | | GERAFKAWAVARLSQRFPKAEFAEVSKLVTDLTKVHTECC |
| | | HGDLLECADDRADLAKYICENQDSISSKLKECCEKPLLEKS |
| | | HCIAEVENDEMPADLPSLAADFVESKDVCKNYAEAKDVFL |
| | | GMFLYEYARRHPDYSVVLLLRLAKTYETTLEKCCAAADPH |
| | | ECYAKVFDEFKPLVEEPQNLIKQNCELFEQLGEYKFQNALL |
| | | VRYTKKVPQVSTPTLVEVSRNLGKVGSKCCKHPEAKRMPC |
| | | AEDYLSVVLNQLCVLHEKTPVSDRVTKCCTESLVNRRPCFS |
| | | ALEVDETYVPKEFNAETFTFHADICTLSEKERQIKKQTALVE |
| | | LVKHKPKATKEQLKAVMDDFAAFVEKCCKADDKETCFAE |
| | | EGKKLVAASQAALGL |
| 2 | CD40L- | IEVKDVTDTTALITWSDDFGEYVWCELTYGIKDVPGDRTTI |
| | specific Tn3 | DLWYHHAHYSIGNLKPDTEYEVSLICRRGDMSSNPAKETFT |
| | monomer with | T |
| | affinity mature | |
| | variant | |
| | Clone 342- | |
| | Affinity | |
| | mature variant | |
| | (W/WT FG | |
| | loop; W/O N- | |
| | Term A, C- | |
| | Term Linker | |
| | and His8Tag | |
| 3 | CD40L- | IEVKDVTDTTALITWSDDFGEYVWCELTYGIKDVPGDRTTI |
| | specific Tn3 | DLWYHHAHYSIGNLKPDTEYEVSLICRSGDMSSNPAKETFT |
| | monomer with | T |
| | affinity mature | |
| | variant | |
| | Clone 342- | |
| | Affinity | |
| | mature variant | |
| | (W/FG loop | |
| | variant RR→ | |
| | RS underlined) | |
| 4 | Human serum | DAHKSEVAHRFKDLGEENFKALVLIAFAQYLQQSPFEDHVK |
| | albumin | LVNEVTEFAKTCVADESAENCDKSLHTLFGDKLCTVATLRE |
| | variant | TYGEMADCCAKQEPERNECFLQHKDDNPNLPRLVRPEVDV |
| | | MCTAFHDNEETFLKKYLYEIARRHPYFYAPELLFFAKRYKA |
| | | AFTECCQAADKAACLLPKLDELRDEGKASSAKQRLKCASL |
| | | QKFGERAFKAWAVARLSQRFPKAEFAEVSKLVTDLTKVHT |
| | | ECCHGDLLECADDRADLAKYICENQDSISSKLKECCEKPLLE |
| | | KSHCIAEVENDEMPADLPSLAADFVESKDVCKNYAEAKDV |
| | | FLGMFLYEYARRHPDYSVVLLLRLAKTYETTLEKCCAAAD |
| | | PHECYAKVFDEFKPLVEEPQNLIKQNCELFEQLGEYKFQNA |
| | | LLVRYTKKVPQVSTPTLVEVSRNLGKVGSKCCKHPEAKRM |
| | | PCAEDYLSVVLNQLCVLHEKTPVSDRVTKCCTESLVNRRPC |
| | | FSALEVDETYVPKEFNAETFTFHADICTLSEKERQIKKQTAL |
| | | VELVKHKPKATKEQLKAVMDDFAAFVEKCCKADDKETCF |
| | | AEEGKKLVAASQAALGL |
| 5 | beta strand “A” | IEV |
| | within a | |
| | CD40L- | |
| | specific | |
| | monomer | |
| 6 | beta strand “B” | ALITW |
| | within a | |
| | CD40L- | |
| | specific | |
| | monomer | |
| 7 | Linker | GGGGSGGGGS |
| 8 | Linker | GGGGSGGGGSGGGGS |
| 9 | Linker | GGGGGGGGGG |
| 10 | Linker | GGGGGGGGGGGGGGG |
| 11 | AB loop | KDVTDTT |
| 12 | BC loop | SDDFGEYVW |
| 13 | CD loop | KDVPGDR |
| 14 | DE loop | WYHHAH |
| 15 | EF loop | GNLKPDTE |
| 16 | FG loop | RSGDMSSNPA |
| 17 | beta strand “C” | CELTYGI |
| | within the | |
| | CD40L- | |
| | specific | |
| | monomer | |
| 18 | beta strand “D” | TTIDL |
| | within a | |
| | CD40L- | |
| | specific | |
| | monomer | |
| 19 | beta strand “E” | YSI |
| | within a | |
| | CD40L- | |
| | specific | |
| | monomer | |
| 20 | beta strand “F” | YEVSLIC |
| | within a | |
| | CD40L- | |
| | specific | |
| | monomer | |
| 21 | beta strand “G” | KETFTT |
| | within a | |
| | CD40L- | |
| | specific | |
| | monomer | |
| 22 | CD40L- | SQIEVKDVTDTTALITWSDDFGEYVWCELTYGIKDVPGDRT |
| | specific Tn3 | TIDLWYHHAHYSIGNLKPDTEYEVSLICRSGDMSSNPAKETF |
| | monomer | TT |
| | Clone 342- | |
| | variant | |
| | (W/FG loop | |
| | variant RR→ | |
| | RS underlined) | |
| 23 | beta strand | RLDAPSQIEV |
| | sequence “A” | |
| | within a | |
| | CD40L- | |
| | specific | |
| | monomer | |
| 24 | beta strand “A” | SQIEV |
| | within a | |
| | CD40L- | |
| | specific | |
| | monomer | |

If the Tn3 scaffold has amino acid sequence changes relative to those shown in SEQ ID NO: 1, the changes may be to the amino acid sequence of one or both of the CD40L-specific Tn3 monomers, so long as it does not adversely effect in vivo efficacy of the Tn3 scaffold, e.g., change in amino acid sequence such that one or both CD40L-specific Tn3 monomers have the amino acid sequence as shown in SEQ ID NO: 2, SEQ ID NO: 3, and SEQ ID NO: 22. In aspects, the first one or two N-terminal amino acid residues (SQ) may be absent and/or substituted with alternative amino acid residues.

In aspects, a Tn3 scaffold comprises at least one CD40L-specific monomer subunit bound to a heterologous moiety. In aspects, this heterologous moiety is selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of said moieties.

In aspects, the heterologous moiety is an antibody. In aspects, the antibody is selected from the group consisting of: an Fc domain of an antibody, an antibody fragment, and a single chain antibody. In aspects, the heterologous moiety is an imaging agent; for example, a radionuclide or biotin. In aspects, the heterologous moiety is a drug; for example, a cytotoxic agent or a radioactive compound.

In aspects, the Tn3 scaffold comprises at least one (e.g. two) CD40L-specific monomer subunit fused or conjugated directly or via a linker to PEG. In aspects, the Tn3 scaffold comprises at least one CD40L-specific monomer subunit fused or conjugated directly or via a linker to an albumin. In aspects, this albumin is human serum albumin (HSA). In aspects, this HSA is a variant HSA. In aspects, the amino acid sequence of the variant HSA is SEQ ID NO: 4. In aspects, the variant HSA has at least one improved property compared with a native HSA or a native HSA fragment. In aspects, the improved property is an altered plasma half-life compared with the plasma half-life of a native HSA or a native HSA fragment. In aspects, the altered plasma half-life is a longer plasma half-life compared with the plasma half-life of a native HSA or a native HSA fragment. In aspects, the altered plasma half-life is a shorter plasma half-life compared with the plasma half-life of a native HSA or a native HSA fragment.

Methods of Treatment

In aspects herein, methods are directed to treating lupus nephritis by administering a Tn3 scaffold comprising a CD40L-specific monomer subunit. In aspects, the CD40L-specific monomer submit is administered in combination with at least one other therapy. In aspects, the at least one other therapy can be a standard-of-care therapy. In aspects, the Tn3 scaffold binds to CD40L. In aspects, the monomer subunit of the Tn3 scaffold comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16. In aspects, the Tn3 scaffold is VIB4920 (i.e., SEQ ID NO:1).

In aspects, the Tn3 scaffold is administered in combination with an immunosuppressant. In aspects, a subject is administered a preparative regimen comprising at least one immunosuppressant in an amount sufficient to reduce an immune response in the subject. In aspects, an immune response is ascertained by determining levels of one or more of: de novo purine synthesis, a cytokine (e.g., IL-2 or IFN-gamma), complement in the blood, immunoglobulin, lymphocyte count, white blood cell count, and combinations thereof.

Any immunosuppressant may be utilized in a treatment regimen of the disclosure. In aspects, the immunosuppressant is selected from the group consisting of: Azathioprine, Mycophenolate mofetil, Cyclosporine, Methotrexate, Leflunomide, Cyclophosphamide, Chlorambucil, Nitrogen mustard, and combinations thereof. In aspects, the immunosuppressant is Azathioprine, Mycophenolate mofetil, or cyclophosphamide. In aspects, the immunosuppressant is mycophenolate mofetil (MMF). In aspects, the Tn3 scaffold is administered in combination with MMF and methylprednisolone. In a particular aspect, the Tn3 scaffold is VIB4920. In aspects, the Tn3 scaffold is administered in combination with cyclophosphamide. In a particular aspect, the Tn3 scaffold is VIB4920.

In aspects, MMF is administered for at least about eight, nine, ten, eleven, twelve or more weeks prior to a first administration of VIB4920. In aspects, if a subject has a response to MMF, the subject is not treated with VIB4920. In aspects, if a subject has a response to MMF by week 8 of treatment with MMF, that subject is not treated with VIB4920.

In aspects, the Tn3 scaffold is administered in combination with MMF or cyclophosphamide and one or more additional therapies. In aspects, the Tn3 scaffold is administered in combination with MMF or cyclophosphamide and prednisone. In aspects, the Tn3 scaffold is administered in combination with MMF and prednisone. In aspects, the Tn3 scaffold is administered in combination with cyclophosphamide and prednisone. In aspects, the Tn3 scaffold is administered in combination with any standard of care therapy for lupus nephritis. In a particular aspect, the Tn3 scaffold is VIB4920.

In aspects, provided is a method comprising administering to subject with lupus nephritis a preparative regime that comprises administration of at least one immunosuppressant in an amount sufficient to reduce an immune response in the subject; and about 1000-2000 mg of VIB4920. In aspects, VIB4920 is administered at a dose from about: 1000-2000 mg, 1200-1800 mg, 1400-1600 mg, or 1450-1550 mg. In aspects, the VIB4920 is administered at a dose of about 1400 mg, 1450 mg, 1500 mg, 1550 mg, or 1600 mg. The VIB4920 can be administered according to any of the schedules provided herein. In aspects, VIB4920 is administered once about every 2 weeks for at least 2 doses, and is administered about once a month thereafter.

The treatment of the autoimmune disease or disorder may be in the form of suppressing a B cell- or T cell-mediated immune response, which may be a reduction of class-switched antibodies, a reduction in circulating B cell subsets, a reduction in plasma activity or a reduction in plasma cells and plasma cell gene signature. The treatment of the autoimmune disease or disorder may be a reduction in markers of inflammation. The markers of inflammation may be one or more of autoantibody levels, plasma cell (PC) or PC gene signature (signature characterized by expression of genes IGHA1, IGJ, IGKC, IGKV4-1 and TNFRSF17), circulating B cell subsets and class-switched antibodies. The treatment of the autoimmune disease or disorder may be a reduction of clinical signs and symptoms, such as those measured by a subject or physician global assessment. Clinical signs and symptoms may include one or more of arthritis, pain, fatigue, fever, malaise, rash, weakness, or signs of organ dysfunction such as proteinuria or loss of kidney function.

In aspects, the treatment of lupus nephritis may be characterized by a reduction of at least about 10%, about 20%, about 30%, about 40%, about 50% or more of clinical symptoms of the disease or disorder, or by a reduction in inflammation, or by a reduction in biomarkers of the disease or disorder, relative to their levels prior to the treatment with VIB4920. The reduction of any of these symptoms, or inflammation, or biomarkers, may be a reduction in the symptoms, or inflammation or biomarkers of at least about 25%, about 30%, about 40%, about 50%, about 60%, about 70%, about 75%, or more relative to their levels prior to the initiation of treatment with VIB4920. The reduction may be such that the autoimmune disease or disorder is characterized as being in remission. If VIB4920 is used in a method of reducing inflammation in an inflammatory disease or disorder, the inflammatory disease or disorder may be lupus nephritis, cutaneous lupus, or SLE.

In aspects, any of the methods disclosed herein result in an improvement of the urinary protein/creatinine ratio (UPCR) of at least about 10%, at least about 15%, at least about 20%, at least about 25%, at least about 30%, at least about 35%, at least about 40%, at least about 45%, at least about 50%, at least about 55%, at least about 60%, at least about 70%, at least about 75%, or greater as compared to a 24-hour baseline measurement in the treated subject.

In aspects, any of the methods disclosed herein result in an improvement of the eGFR (glomerular filtration rate) of at least about 10%, at least about 15%, at least about 20%, at least about 25%, at least about 30%, at least about 35%, at least about 40%, at least about 45%, at least about 50%, at least about 55%, at least about 60%, at least about 70%, at least about 75%, at least about 80%, at least about 85%, at least about 90% or greater as compared to a baseline measurement in the treated subject. In aspects, any of the methods disclosed herein result in an improvement of the eGFR (glomerular filtration rate) to ≥120 ml/min/1.73 m².

In aspects, a subject receiving treatment comprises a subject having an autoimmune disorder. In aspects, an autoimmune disorder comprises LN, Systemic Lupus Erythematosus (SLE), renal failure, renal disease, and combinations thereof. In aspects, a subject is an adult subject. In aspects, a subject is pediatric. In aspects, a subject comprises SLE as determined by the 1997 update of the 1982 American College of Rheumatology (ACR) criteria, the 2012 Systemic Lupus International Collaborating Clinics (SLICC) criteria, or the 2019 European League Against Rheumatism (EULAR)/ACR criteria. In aspects, a subject has a UPCR ≥1.5 based on a 24-hour urine collection. In aspects, a subject has a renal biopsy resulting in ISN/RPS LN of a class selected from the group consisting of: Class III, Class IV, or Class V in combination with Class III or IV, and modified NIH activity index ≥1 (Bajema I M et al. Revision of the International Society of Nephrology/Renal Pathology Society classification for lupus nephritis: clarification of definitions and modified National Institutes of Health activity and chronicity indices. Kidney international. 2018: 93(4):789-96).

Dosing

The dose of a Tn3 scaffold (e.g., VIB4920) administered in any of the methods disclosed herein may be a dose of between about 500 mg and about 3000 mg. In aspects, the Tn3 scaffold is administered at a dose from about: 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, 4800 mg, 4850 mg, 4900 mg, 4950 mg, or about 5000 mg. In aspects, the Tn3 scaffold is administered at a dose of between about 1500 mg and about 3000 mg. In aspects, the Tn3 scaffold is administered at a dose selected from the group consisting of: 1500 mg and 3000 mg. In aspects, the Tn3 scaffold is administered at a dose of about 1500 mg. In aspects, the Tn3 scaffold is administered at a dose of about 3000 mg. In aspects, Tn3 scaffold may be administered at a dose of about: 500 mg, about 750 mg, about 900 mg, about 1000 mg, about 1250 mg, about 1500 mg, about 1750 mg, about 2000 mg, about 2250 mg, about 2500 mg, or about 3000 mg. For example, Tn3 scaffold is administered at a dose of about 1500 mg.

In aspects, a Tn3 scaffold is administered at a dose of 1500 mg intravenously for 8 doses at weeks 10, 12, 14, 18, 22, 26, 30, and 34, according to any of the schedules provided herein.

In aspects, the Tn3 scaffold used in any of the methods disclosed herein may be administered about every other week or may be administered twice per month. In aspects, Tn3 scaffold may be administered about once a month. In aspects, Tn3 scaffold is administered once about every 2 weeks for at least 2, 3, 4, 5, 6 or more doses, and is administered about once a month thereafter. In aspects, Tn3 scaffold is administered once about every 2 weeks for at least 2 doses, and is administered about once a month thereafter. In aspects, Tn3 scaffold is administered once about every 2 weeks for at least 3 doses, and is administered about once a month thereafter. Tn3 scaffold may be administered by intravenous or subcutaneous injection. In aspects, Tn3 scaffold is administered by intravenous injection. In aspects, Tn3 scaffold is administered by intravenous infusion.

In aspects, the description provides a method for treating lupus nephritis in a subject in need thereof comprising: administering a Tn3 scaffold to the subject; wherein the Tn3 scaffold is administered at a dose of about 1500 mg, and wherein Tn3 scaffold is administered once about every 2 weeks for at least 2 doses, and is administered about once a month thereafter. In aspects, the description provides a method for treating lupus nephritis in a subject in need thereof comprising: administering a Tn3 scaffold to the subject; wherein the Tn3 scaffold is administered at a dose of about 1500 mg, and wherein the Tn3 scaffold is administered once about every 2 weeks for at least 3 doses, and is administered about once a month thereafter. In aspects, prednisone is additionally administered in methods disclosed herein.

In any of the methods disclosed herein, prednisone is administered at a standard-of-care dose. In aspects, prednisone is administered at a dose of about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 21 mg/day, about 22 mg/day, about 23 mg/day, about 24 mg/day, about 25 mg/day, about 26 mg/day, about 27 mg/day, about 28 mg/day, about 29 mg/day, about 30 mg/day, about 35 mg/day, about 40 mg/day, or about 50 mg/day. In aspects, prednisone is administered at a dose of 25 mg/day. In aspects, the administration of prednisone is tapered off, such that a higher dose is administered initially and then reduced. The tapering off can be performed daily, weekly, or monthly. In aspects, the tapering is performed weekly. The tapering off can comprise reducing an administration of prednisone from any range from 50 mg/day down to 1 mg/day. In aspects, an administration of prednisone is tapered from an initial administration of 25 mg/day and tapered off to 5 mg/day. In aspects, an administration of prednisone is tapered from an initial administration of 25 mg/day at Day 0, or on the day after completion of methylprednisolone and tapered off to 5 mg/day at week 8.

In any of the methods disclosed herein, methylprednisolone is administered at a standard-of-care dose. In aspects, methylprednisolone is administered at a dose of about 1 mg/day, about 5 mg/day, about 10 mg/day, about 20 mg/day, about 30 mg/day, about 50 mg/day, about 100 mg/day, about 150 mg/day, about 200 mg/day, about 250 mg/day, about 300 mg/day, about 350 mg/day, about 400 mg/day, about 500 mg/day, about 600 mg/day, about 700 mg/day, about 800 mg/day, about 900 mg/day, about 1000 mg/day, about 1250 mg/day, about 1500 mg/day, or about 2000 mg/day. In aspects, methylprednisolone is administered at a dose of about 1-5 mg/day, about 5-10 mg/day, about 10-20 mg/day, about 20-30 mg/day, about 30-50 mg/day, about 50-100 mg/day, about 100-150 mg/day, about 150-200 mg/day, about 200-250 mg/day, about 250-300 mg/day, about 300-350 mg/day, about 350-400 mg/day, about 400-500 mg/day, about 500-600 mg/day, about 600-700 mg/day, about 700-800 mg/day, about 800-900 mg/day, about 900-1000 mg/day, about 1000-1250 mg/day, about 1250-1500 mg/day, or about 1500-2000 mg/day. In aspects, methylprednisolone is administered at a dose of 500 mg/day. In aspects, methylprednisolone is administered at a dose of 1000 mg/day.

In aspects of the methods disclosed herein, the subject has been administered one or more standard of care therapies for the treatment of lupus prior to the administration of VIB4920. In aspects, the one or more standard of care therapies are administered for about 1, about 2, about 3, about 4, about 5, about 6, about 7, about 8, about 9, about 10, about 11, about 12, about 13, about 14, about 15, about 20, about 25, about 30, about 35, about 40, about 50, about 52 weeks or more prior to the administration of VIB4920.

In aspects, the description provides a method for treating lupus nephritis in a subject in need thereof comprising: administering VIB4920 in combination with mycophenolate mofetil (MMF) to the subject; wherein the VIB4920 is administered at a dose of about 15(0) mg. In aspects, the MMF is administered at any standard-of-care dose. In a specific aspect, MMF is administered at a dose of about 1 gram/day, about 2 grams/day, about 3 grams/day or about 4 grams/day. In one aspect, the MMF is administered at 2 grams/day. In aspects, the MMF is administered at 3 grams/day. In aspects, methylprednisolone is additionally administered. In aspects, prednisone is additionally administered. In aspects, VIB4920 is administered once about every two, three, four weeks, or about once a month. In aspects, VIB4920 is administered once about every 2 weeks for at least 2 doses, and is administered about once a month thereafter. In aspects, VIB4920 is administered once about every 2 weeks for at least 3 doses, and is administered about once a month thereafter. In aspects, the subject has been administered any standard of care therapy for the treatment of lupus prior to the administration of VIB4920. In aspects, the subject has been administered MMF and/or prednisone prior to the administration of VIB4920.

In aspects, the description provides a method for treating lupus nephritis in a subject in need thereof comprising: administering VIB4920 in combination with cyclophosphamide to the subject, wherein VIB4920 is administered at a dose of about 1500 mg. In aspects, the cyclophosphamide is administered at any standard-of-care dose. In aspects, prednisone is additionally administered. In aspects, VIB4920 is administered once about every two, three, four weeks, or about once a month. In aspects, VIB4920 is administered once about every 2 weeks for at least 2 doses, and is administered about once a month thereafter. In aspects, VIB4920 is administered once about every 2 weeks for at least 3 doses, and is administered about once a month thereafter. In aspects, the subject has been administered any standard of care therapy for the treatment of lupus prior to the administration of VIB4920. In aspects, the subject has been administered cyclophosphamide and/or prednisone prior to the administration of VIB4920.

In aspects, VIB4920 is administered sequentially with any therapy disclosed herein. In aspects, VIB4920 is administered concurrently with any therapy disclosed herein. In aspects, the subject has been administered any standard of care therapy for the treatment of lupus prior to the administration of VIB4920.

The dose and dosing regimen of VIB4920 may be such that any therapeutic effect achieved from administration of VIB4920 to treat any autoimmune/inflammatory disease or disorder, may be considered to be “long-lasting.” A “long-lasting” effect of VIB4920 in the treatment of an autoimmune/inflammatory disease or disorder is one in which the therapeutic effect achieved by VIB4920 is maintained (although VIB4920 is no longer administered) over at least 4 weeks, at least 6 weeks, at least 8 weeks, at least 10 weeks, at least 12 weeks, at least 16 weeks, at least 20 weeks, or at least 24 weeks following administration of the last dose of a course of VIB4920.

Response

In aspects, provided are methods that comprise achieving a renal response in a subject with LN by way of administration of VIB4920. A response may be evaluated at any period. In aspects, a response is evaluated before, during, or after administration of VIB4920. In aspects, a response is evaluated after administration of VIB4920. In aspects, a response is evaluated at week 0, 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, or 70 post administration of VIB4920. In aspects, a response is evaluated at week 8.

In aspects, a response is a complete response. In aspects, a complete response comprises achieving a 24-hour UPCR ≤0.5. In aspects, a response comprises achieving a 24-hour UPCR less than about or up to about: 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3. In aspects, a response comprises achieving a 24-hour UPCR less than about or up to about: 0, 0.1, 0.2, 0.3, 0.4, 0.5, 0.6, 0.7, 0.8, 0.9, or 1. In aspects, a complete response comprises achieving a 24-hour UPCR from about 0.1-0.5; 0.1-0.6; or 0.3-0.6.

In aspects, a response comprises achieving an estimated glomerular filtration rate greater than about or up to about: 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 ml/min/1.73 m².

In aspects, a response comprises reducing or eliminating anti-dsDNA antibodies by at least about 5-10 fold, 10-50 fold, 1-20 fold, 1-5 fold, 5-25 fold, 10-40 fold, or 1-10 fold. In aspects, a response comprises reducing or eliminating anti-dsDNA antibodies by at least about: 5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 105 fold, 110 fold, 115 fold, 120 fold, 125 fold, 130 fold, 135 fold, 140 fold, 145 fold, 150 fold, 155 fold, 160 fold, 165 fold, 170 fold, 175 fold, 180 fold, 185 fold, 190 fold, 195 fold, 200 fold, 205 fold, 210 fold, 215 fold, 220 fold, 225 fold, 230 fold, 235 fold, 240 fold, 245 fold, or 250 fold.

In aspects, a complete response also comprises any one of: an inactive urinary sediment as determined by: (a) urinary RBC reported in a range of less than 5-10/hpf, in the absence of menses and infection, (b) urinary WBC reported in a range of less than 5-10/hpf, in the absence of infection, and/or absence of RBC and WBC casts. In aspects, a complete renal response comprises achieving any one of: UPCR ≤0.75, based on a 24-hour collection, an estimated glomerular filtration rate (eGFR) ≥120 ml/min/1.73 m², or if <120 ml/min/1.73 m², then >80% of the eGFR at baseline, and/or prednisone ≤5 mg/day from Week 8, according to the prednisone dosing restrictions described herein.

In aspects, a response comprises achieving any one of: (1) a complete renal response at weeks 26, 48, and 60; and/or an overall renal response at weeks 26, 38, 48, and 60; (2) an overall renal response comprises any one of: ≥50% improvement in the UPCR compared to baseline, based on a 24-hour urine collection, estimated eGFR ≥120 ml/min/1.73 m2, or if <120 ml/min/1.73 m2, then 80% of the eGFR at baseline, and/or prednisone ≤5 mg/day from week 8; (3) UPCR at weeks 26, 38, 48, and 60, based on a 24-hour urine collection; (4) anti-dsDNA antibodies at weeks 26, 38, 48, and 60; (5) C3 levels at weeks 26, 38, 48, and 60; (6) C4 levels at weeks 26, 38, 48, and 60; (7) SLEDAI-2K at weeks 26, 38, 48 and 60; (8) SLICC/ACR-DI at weeks 26 and 60; and/or (9) renal treatment failures.

In aspects, are methods that comprise administering a composition that comprises VIB4920 to a subject in need thereof. In aspects, the subject is administered at least one additional therapeutic. In aspects, the administering is sufficient to reduce at least a symptom of a renal condition. In aspects, the condition comprises LN. In aspects, the symptom comprises muscle pain, fever, rash, swelling, joint pain, high blood pressure, kidney failure, and combinations thereof. In aspects, the administering is sufficient to improve UPCR by at least about 5%, 10%, 15%, 20%, 25%, 30%, 35%, 40%, 45%, 50%, 55%, 60%, 65%, 70%, 75%, 80%, 85%, 90%, 95%, or 100% over baseline or as compared to an otherwise comparable subject lacking the administering.

Formulation

In aspects, VIB4920 is a clear to opalescent, colorless to yellow liquid, free from or practically free from visible particles. VIB4920 can be formulated in various ways and at various concentrations.

In aspects, VIB4920 is formulated from about 10-50 mg/mL, 30-80 mg/mL, 80-100 mg/mL, 90-120 mg/mL, 90-150 mg/mL, 80-150 mg/mL, 100-150 mg/MI, or 50-150 mg/mL. In aspects, VIB4920 is formulated at a concentration from about 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL, to about 200 mg/mL. In aspects, VIB4920 is formulated at 100 mg/mL.

In aspects, a formulation of VIB4920 comprises at least one additional ingredient. In aspects an additional ingredient comprises a buffer. In aspects a buffer is sodium phosphate buffer but other buffers can also be utilized. Sodium phosphate buffer can be formulated at a concentration from about 1-5 mM, 1-10 mM, 2-10 mM, 5-15 mM, 5-10 mM, 8-20 mM, 10-20 mM, or 5-20 mM. In aspects, sodium phosphate is formulated at a concentration from about 1 mM, 2 mM, 3 mM, 4 mM, 5 mM, 6 mM, 7 mM, 8 mM, 9 mM, 10 mM, 1 mM, 12 mM, 13 mM, 14 mM, 15 mM, 16 mM, 17 mM, 18 mM, 19 mM, to about 20 mM. In aspects a formulation of VIB4920 comprises 10 mM of sodium phosphate buffer.

In aspects, a formulation of VIB4920 comprises at least one additional ingredient. In aspects an additional ingredient comprises a sugar. In aspects, a sugar comprises sucrose, but other sugars can also be utilized. Sucrose can be formulated at a concentration from about 50-100 mM, 75-125 mM, 100-200 mM, 150-250 mM, 150-300 mM, 200-300 mM, 225-325 mM, or 230-280 mM. In aspects, sucrose is formulated at a concentration from about 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, or up to about 300 mM. In aspects, a formulation of VIB4920 comprises 250 mM sucrose.

In aspects, a formulation of VIB4920 comprises at least one additional ingredient. In aspects an additional ingredient comprises a surfactant. A surfactant can be useful as an antifoaming agent, wetting agent, dispersant, thickener, and/or emulsifier in a formulation. In aspects, a surfactant comprises poloxamer 188, but other surfactants can also be utilized, poloxamer 188 can be formulated at a concentration from about 0-0.02% weight/volume, 0.01-0.02% weight/volume, 0-1% weight/volume, or 0.001-0.05% weight/volume. In aspects, poloxamer 188 is formulated at a concentration from about 0% weight/volume, 0.1% weight/volume, 0.2% weight/volume, 0.3% weight/volume, 0.4% weight/volume, 0.5% weight/volume, 0.6% weight/volume, 0.7% weight/volume, 0.8% weight/volume, 0.9% weight/volume, 1% weight/volume. In aspects, a formulation of VIB4920 comprises 0.02% weight/volume poloxamer 188. Any of the abovementioned concentrations can be taken at a pH that is basic, neutral, or acidic. In aspects, the pH is neutral. In aspects the pH is from 6-8. In aspects, the pH is from 6.5-7.5. In aspects the pH is 7. In aspects the pH is 7+/1.5. In aspects the pH is 7.4. In aspects, a formulation of VIB4920 comprises 0.02% weight/volume poloxamer 188 at pH 7.4. In aspects, a composition comprises VIB4920 at 100 mg/mL in 10 mM sodium phosphate buffer, 250 mM sucrose, and 0.02% weight/volume poloxamer 188 pH 7.4. In aspects, a vial of VIB4920 contains 500 mg in 5 mL.

In aspects, preparation of a formulation of VIB4920 can be done in multiple ways. In aspects, VIB4920 can be equilibrated to room temperature for approximately 15 minutes, but no longer than 2 hours prior to preparation of the formulation. VIB4920 vials can be evaluated, prior to preparation of the formulation, to ensure that there are no particles, or reduced particles, and the color is not different from the description as previously described.

In aspects, three vials of VIB4920 containing 5 mL each will be used to prepare a dose of 1500 mg VIB4920. In aspects, 15 ml can be removed from a 250 mL bag of 0.9% normal saline, and replaced with 15 mL VIB4920 (5 mL from each of 3 vials). The contents of the bag can be mixed gently by inverting, but should not be shaken. If discoloration or particles are observed, the bag should not be administered.

Diluted VIB4920 may be stored prior to administration, refrigerated at 2° C. to 8° C. for a maximum of 24 hours from the initial puncture of the VIB4920 vial, or at room temperature for a maximum of 4 hours from the initial puncture of the VIB4920 vial.

Provided herein is also a pharmaceutical composition that comprises VIB4920. In aspects, a pharmaceutical composition is in unit dosage form. In aspects, a pharmaceutical composition comprises a pharmaceutically acceptable excipient. Exemplary excipients can include dextrose, sodium chloride, sucrose, lactose, cellulose, xylitol, sorbitol, malitol, gelatin, PEG, PVP, and any combination thereof. In aspects, a pharmaceutical composition comprises VIB4920 and at least one of a buffer, sugar, surfactant, and combinations thereof.

In aspects, an excipient is selected from the group consisting of: sodium phosphate buffer, sucrose, poloxamer 188, and combinations thereof. In aspects, a formulation of VIB4920 comprises about 100 mg/mL of VIB4920 in 10 mM sodium phosphate buffer, 250 mM sucrose, and 0.02% weight/volume poloxamer 188 pH 7.4. In aspects, a vial of VIB4920 contains 500 mg in 5 mL.

Administration

Provided herein is also administration of a pharmaceutical composition that comprises VIB4920. In aspects, a pharmaceutical composition is administered to a subject in need thereof. An administration can be prophylactic. In aspects, an administration is effective to treat a subject in need thereof. In aspects, an administration is effective in reducing or eliminating an adverse event.

In some instances, a pharmaceutical composition that comprises VIB4920 is administered by a route selected from subcutaneous injection or infusion, intramuscular injection or infusion, intradermal injection or infusion, percutaneous administration, intravenous (“i.v.”) administration, intranasal administration, intralymphatic injection or infusion, and oral administration. In some instances, a subject is infused with a pharmaceutical composition comprising VIB4920 by an intralymphatic microcatheter. In aspects, a pharmaceutical composition comprising VIB4920 is administered intravenously via infusion pump.

In aspects, an administration of a pharmaceutical composition is performed over a period of time. In aspects, an administration is performed by the minute, hourly, daily, weekly or monthly. In aspects, an administration is performed over a period of time from about 5 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, 120 min, 125 min, 130 min, 135 min, 140 min, 145 min, 150 min, 155 min, 160 min, 165 min, 170 min, 175 min, 180 min, 185 min, 190 min, 195 min, up to about 200 min. In aspects, an administration is performed over a period of 90 minutes.

Kits

Provided herein are also kits that comprise a Tn3 scaffold. In aspects, VIB4920 is comprised in a container. Suitable containers comprise vials, tubes, syringes, bags, and combinations thereof. In aspects, containers are frozen. In aspects, contains are not frozen. In aspects, a container that comprises VIB4920 is stored at 2° C. to 8° C. (36° F. to 46° F.).

In aspects, VIB4920 is comprised in a vial. A vial can comprise any amount of VIB4920. In aspects, a vial comprises from about 0.5-1 mL, 0.5-1.5 mL, 1-2 mL, 2-4 mL, 1-5 mL, 5-10 mL, or 1-10 mL. In aspects a vial comprises 5 mL of VIB4920. A vial of VIB4920 can comprise a concentration from about 10-50 mg, 50-100 mg, 100-300 mg, 200-500 mg, 300-500 mg, 300-600 mg, 350-650 mg, 400-800 mg, 400-1000 mg, or 500-1000 mg. In aspects, a vial comprises about 500 mg of VIB4920. In aspects, a vial of VIB4920 contains 500 mg in 5 mL. In aspects, VIB4920 is comprised in a container that contains 500 mg in 5 mL of VIB4920.

In aspects, a kit or container that comprises VIB4920 is not shaken during any step provided herein.

In aspects, a kit or container that comprises VIB4920 is labeled.

Those skilled in the art will recognize, or be able to ascertain using no more than routine experimentation, many equivalents to the specific aspects described herein. Such equivalents are intended to be encompassed by the following claims.

### Embodiments

1. A method for treating lupus nephritis in a subject in need thereof comprising: administering a Tn3 scaffold comprising a CD40L-specific monomer subunit to the subject, wherein the Tn3 scaffold binds to CD40L, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 weeks for at least 2 doses, and is administered about once a month thereafter.

2. A method for treating lupus nephritis in a subject in need thereof comprising: administering a Tn3 scaffold comprising a CD40L-specific monomer subunit to the subject, wherein the Tn3 scaffold binds to CD40L, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the Tn3 scaffold comprising the CD40L-specific monomer subunit is administered at a dose of about 1500 mg, and wherein the Tn3 scaffold is administered once about every 2 weeks for at least 3 doses, and is administered about once a month thereafter.

3. A method for treating lupus nephritis in a subject in need thereof comprising: administering a Tn3 scaffold comprising a CD40L-specific monomer subunit in combination with mycophenolate mofetil (MMF) to the subject, wherein the Tn3 scaffold binds to CD40L, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, and wherein the Tn3 scaffold is administered at a dose of about 1500 mg.

4. A method for treating lupus nephritis in a subject in need thereof comprising: administering a Tn3 scaffold comprising a CD40L-specific monomer subunit in combination with cyclophosphamide to the subject, wherein the Tn3 scaffold binds to CD40L, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, and wherein the Tn3 scaffold is administered at a dose of about 1500 mg.

5. The method of embodiment 4, wherein the cyclophosphamide is administered for at least about eight, ten, twelve or more weeks prior to the administration of the Tn3 scaffold.

6. The method of embodiment 5, wherein the cyclophosphamide is administered for at least about eight weeks prior to the administration of the Tn3 scaffold.

7. The method of embodiment 5, wherein the cyclophosphamide is administered for at least about ten weeks prior to the administration of the Tn3 scaffold.

8. The method of embodiment 5, wherein the cyclophosphamide is administered for at least about twelve weeks prior to the administration of the Tn3 scaffold.

9. The method of any one of embodiments 3-8, wherein the Tn3 scaffold is administered once about every two, three, four weeks, or about once a month.

10. The method of any one of embodiments 3-8, wherein the Tn3 scaffold is administered once about every 2 weeks for at least 2 doses, and is administered about once a month thereafter.

11. The method of any one of embodiments 3-8, wherein the Tn3 scaffold is administered once about every 2 weeks for at least 3 doses, and is administered about once a month thereafter.

12. The method of any of embodiments 1-11, wherein the Tn3 scaffold is administered intravenously.

13. The method of any one of embodiments 1-12, wherein the Tn3 scaffold comprises two CD40L-specific monomer subunits connected in tandem.

14. The method of any one of embodiments 1-13, wherein the Tn3 scaffold: a. binds CD40L thereby reducing or preventing binding of CD40L to CD40; b. reduces or eliminates CD40 mediated signaling; or c. a and b.

15. The method of any one of embodiments 1-14, wherein at least one CD40L-specific monomer subunit is fused or conjugated to a heterologous moiety selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, and a cytokine.

16. The method of any one of embodiments 13-15, wherein at least one of the two CD40L-specific monomer subunits is conjugated to PEG or is fused to a human serum albumin (HSA).

17. The method of embodiment 16, wherein the HSA is a variant HSA comprising the amino acid sequence of SEQ ID NO: 4.

18. The method of any one of embodiments 1-17, wherein the Tn3 scaffold comprises the sequence of SEQ ID NO: 1.

19. The method of any one of embodiments 1-18, wherein the Tn3 scaffold is VIB4920.

20. The method of any one of embodiments 1-19, wherein the subject is further administered prednisone.

21. The method of any one of claims 1-20, wherein the subject is further administered methylprednisolone.

22. The method of any one of embodiments 1-21, wherein the subject receives one or more standard of care therapies prior to the administration of the Tn3 scaffold.

23. The method of embodiment 22, wherein the standard of care therapy is mycophenolate mofetil (MMF).

24. The method of embodiment 22, wherein the standard of care therapy is cyclophosphamide.

25. The method of embodiment 22, wherein the standard of care therapy is prednisone.

26. The method of claim 22, wherein the standard of care therapy is methylprednisolone.

27. The method of any one of claims 1-26, wherein the subject has active lupus, optionally wherein the subject has a modified NIH activity index ≥1.

28. A method for reducing or treating an autoimmune condition in a subject in need thereof, the method comprising administering a composition that comprises a Tn3 scaffold comprising a CD40L-specific monomer subunit, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, wherein the administering is effective in reducing the autoimmune condition as determined by at least one of: a. a urine protein/creatinine ratio from 0.7-0.8 based on a 24 hour urine collection; b. an estimated glomerular filtration rate of greater than about 120 ml/min/1.73 m2; c. an estimated glomerular filtration rate of greater than about 80% of the glomerular filtration rate prior to the administering; d. prednisone administration of less than about 5 mg/day after the administering; or e. any combination of a −d.

29. The method of embodiment 28, comprising a preparatory regime that comprises administration of at least one therapeutic prior to the administering.

30. The method of embodiment 29, wherein the at least one therapeutic comprises an immunosuppressant.

31. The method of embodiment 29, wherein the at least one therapeutic comprises a steroid.

32. The method of any one of embodiments 29-31, wherein the at least one therapeutic comprises an immunosuppressant and a steroid.

33. The method of embodiment 32, wherein the immunosuppressant comprises Mycophenolate mofetil (MMF).

34. The method of embodiment 33, wherein the MMF is administered at a dose from about 2-3 g per day.

35. The method of embodiment 32, wherein the steroid comprises prednisone.

36. The method of embodiment 32, wherein the steroid comprises methylprednisolone.

37. The method of embodiment 35, wherein the prednisone is administered at a dose from about 5 mg per day.

38. The method of any one of embodiments 28-37, wherein the composition comprises about 1500 mg of the Tn3 scaffold comprising a CD40L-specific monomer subunit.

39. The method of any one of embodiments 28-38, wherein the administering is intravenous.

40. The method of any one of embodiments 32-38, wherein the administering is concurrent with the immunosuppressant and the steroid.

41. The method of any one of embodiments 32-40, wherein the autoimmune condition comprises Systemic Lupus Erythematosus.

42. The method of any one of embodiment 32-41, wherein prior to the administering, the subject comprises a urine protein/creatinine ratio greater than about 1.5 based on a 24-hour urine collection.

43. A method of improving renal function in a Systemic Lupus Erythematosus positive subject, the method comprising administering a pharmaceutical composition that comprises a Tn3 scaffold comprising a CD40L-specific monomer subunit at 1500 mg to the subject, wherein the subject has a urine protein/creatinine ratio in a range of greater than 1.5, wherein the subject is also administered Mycophenolate mofetil (MMF) and prednisone, wherein the MMF is not more than 3 g/day, and wherein the prednisone is not more than 5 mg/day.

44. The method of embodiment 43, wherein the renal function is improved as determined by: a. a urine protein/creatinine ratio from 0.7-0.8 based on a 24 hour urine collection; b. an estimated glomerular filtration rate of greater than about 120 ml/min/1.73 m2; c. an estimated glomerular filtration rate of greater than about 80% of the glomerular filtration rate prior to the administering; d. prednisone administration of less than about 5 mg/day after the administering; or e. any combination of a −d.

45. The method of any one of embodiments 43-44, wherein the Systemic Lupus Erythematosus positive subject has Lupus nephritis.

46. The method of any one of embodiments 43-45, wherein the MMF is administered at a dose from about 2-3 g per day.

47. The method of any one of embodiments 43-46, wherein the prednisone is administered at 5 mg per day.

48. The method of any one of embodiments 43-47, wherein the MMF and the prednisone are administered prior to the Tn3 scaffold comprising a CD40L-specific monomer subunit.

49. The method of any one of embodiments 4347, wherein the MMF and the prednisone are administered concurrent with the Tn3 scaffold comprising a CD40L-specific monomer subunit.

50. The method of any one of embodiments 43-49, wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16.

51. The method of any one of embodiments 43-50, wherein the subject is treatment-naïve with respect to the Tn3 scaffold comprising a CD40L-specific monomer subunit.

52. The method of any of the preceding embodiments, wherein the beta A strand comprises SEQ ID NO: 5, SEQ ID NO: 23, or SEQ ID NO: 24, wherein the beta B strand comprises SEQ ID NO: 6, wherein the beta C strand comprises SEQ ID NO: 17, wherein the beta D strand comprises SEQ ID NO: 18, wherein the beta E strand comprises SEQ ID NO: 19, wherein the beta F strand comprises SEQ ID NO: 20, and wherein the beta G strand comprises SEQ ID NO: 21.

53. The method of embodiment 52, wherein the beta A strand consists of SEQ ID NO: 5.

54. The method of embodiment 52, wherein the beta A strand consists of SEQ ID NO: 23.

55. The method of embodiment 52, wherein the beta A strand consists of SEQ ID NO: 24.

56. The method of embodiment 52, wherein the beta B strand consists of SEQ ID NO: 6.

57. The method of embodiment 52, wherein the beta C strand consists of SEQ ID NO: 17.

58. The method of embodiment 52, wherein the beta D strand consists of SEQ ID NO: 18.

59. The method of embodiment 52, wherein the beta E strand consists of SEQ ID NO: 19.

60. The method of embodiment 52, wherein the beta F strand consists of SEQ ID NO: 20.

61. The method of embodiment 52, wherein the beta G strand consists of SEQ ID NO: 21.

### Embodiment Set 2

1. A method for treating lupus nephritis in a subject in need thereof comprising: administering Dazodalibep to the subject, wherein the Dazodalibep is administered at a dose of about 1500 mg, wherein the Dazodalibep is administered once about every 2 weeks for at least 2 doses, and is administered about once a month thereafter.

2. A method for treating lupus nephritis in a subject in need thereof comprising: administering a dose of about 1500 mg Dazodalibep to the subject, wherein the Dazodalibep is administered one about every 2 weeks for 3 doses, and is administered about once a month thereafter.

3. A method for treating lupus nephritis in a subject in need thereof comprising: administering Dazodalibep in combination with mycophenolate mofetil (MMF) to the subject, wherein the Dazodalibep is administered at a dose of about 1500 mg.

4. The method of embodiment 3, wherein the MMF is administered for at least about eight, nine, ten, eleven, twelve or more weeks prior to the first administration of the Dazodalibep.

5. A method for treating lupus nephritis in a subject in need thereof comprising: administering Dazodalibep in combination with cyclophosphamide to the subject, wherein the Dazodalibep is administered at a dose of about 1500 mg.

6. The method of embodiment 5, wherein the cyclophosphamide is administered for at least about eight, nine, ten, eleven, twelve or more weeks prior to a first administration of the Dazodalibep.

### Embodiment Set 3

1. A method of treating lupus nephritis, the method comprising administering to a subject in need thereof: a) a preparative regime that comprises administration of at least one immunosuppressant in an amount sufficient to reduce an immune response in the subject; and b) about 1000-2000 mg of Dazodalibep, wherein the Dazodalibep is administered once about every 2 weeks for at least 2 doses, and is administered about once a month thereafter.

2 A method of treating lupus nephritis, the method comprising administering to a subject in need thereof: a) a preparative regime that comprises: i. administration of an immunosuppressant in an amount sufficient to reduce an immune response in the subject; and ii. administration of a corticosteroid in an amount sufficient to reduce inflammation in the student; and b) about 1000-2000 mg of Dazodalibep, wherein the Dazodalibep is administered once about every 2 weeks for at least 2 doses, and is administered about once a month thereafter.

3. The method of any one of embodiments 1-2, wherein the immunosuppressant is selected from the group consisting of: Azathioprine, Mycophenolate mofetil, Cyclosporine, Methotrexate, Leflunomide, Cyclophosphamide, Chlorambucil, Nitrogen mustard, and combinations thereof.

4. The method of embodiment 3, wherein the immunosuppressant is selected from the group consisting of: azathioprine, mycophenolate mofetil, cyclophosphamide, and cyclosporine.

5. The method of embodiment 3 or 4, wherein the immunosuppressant is mycophenolate mofetil.

6. The method of embodiment 4 or 5, wherein the immunosuppressant is cyclophosphamide.

7. The method of any one of embodiments 25-30, wherein the immunosuppressant is administered for at least about eight, nine, ten, eleven, twelve or more weeks prior to a first administration of the Dazodalibep.

8. The method of any one of embodiments 6-7, wherein the cyclophosphamide is administered every 4 weeks.

9. The method of any one of embodiments 6-7, wherein the cyclophosphamide is administered every 2 weeks.

10. The method of any one of embodiments 1-9, wherein about 1200-1800 mg of the Dazodalibep is administered.

11. The method of embodiment 10, wherein about 1500 mg of the Dazodalibep is administered.

12. The method of embodiment 2, wherein the preparative regimen further comprises administering a corticosteroid to the subject.

13. The method of any one of embodiments 2-12, wherein the corticosteroid is prednisone.

14. The method of any one of embodiments 1-13, wherein the administering of the corticosteroid is tapered.

### EXAMPLES

### Example 1—VIB4920 for Active Lupus Nephritis: A Phase 2a Randomized Placebo-Controlled Double-Blind Multicenter Trial of VIB4920 for Active Lupus Nephritis

A Phase 2a. randomized, double-blind, placebo-controlled multicenter trial of VIB4920 for active lupus nephritis will be conducted in adults age 18 and older classified with Systemic Lupus Erythematosus (SLE) by any of the following criteria: the 1997 update of the 1982 American College of Rheumatology (ACR) criteria, the 2012 Systemic Lupus International Collaborating Clinics (SLICC) criteria, or the 2019 European League Against Rheumatism (EULAR)/ACR criteria, UPCR ≥1.5 based on a 24-hour urine collection at Visit −1 or within 14 days prior to Visit −1, and renal biopsy documentation at Visit −1 of ISN/RPS LN: Class III, Class IV, or Class V in combination with Class III or IV, and modified NIH activity index ≥1.

Study Design

Eligible subjects will undergo induction therapy for LN with doses of MMF, methylprednisolone, and a rapid corticosteroid taper to 5 mg prednisone per day. Subjects will be assessed for an early renal response after 8 weeks of treatment with MMF and corticosteroids, and those with UPCR >0.75 will be randomized to treatment with VIB4920 or placebo. MMF and low dose corticosteroids will be continued.

In brief, up to 114 eligible subjects with active LN will receive induction therapy with MMF and methylprednisolone beginning at Week 0. Subjects will also receive prednisone 25 mg per day beginning at Week 0 and tapered to 5 mg per day at Week 8. Subjects will be assessed at Week 8 for a renal response. Sixty-six subjects with a urine protein-to-creatinine ratio (UPCR) greater than 0.75 will be randomized 2:1 to VIB4920 versus placebo at Week 10. Subjects who are not eligible for randomization will complete study participation after the Week 8 study visit, and further care will be provided according to the judgement of the site investigator or treating physician. Randomized subjects will receive VIB4920 1500 mg or placebo intravenously at Weeks 10, 12, 14, 18, 22, 26, 30, and 34, and will continue MMF 2-3 g per day and prednisone 5 mg per day. The primary endpoint will be assessed at Week 38, and subjects followed until Week 60.

Randomization

Eligible subjects will be randomized 2:1. The criterion of UPCR >0.75 for randomization corresponds to the UPCR component of the primary endpoint definition for complete renal response.

Stratification

Random assignment of subjects to VIB4920 versus placebo will be stratified by proteinuria levels at baseline and Week 8, based on 1) UPCR <3 at baseline, 2) UPCR ≥3 at baseline that has improved by ≥25% at Week 8, and 3) UPCR ≥3 at baseline that has improved by <25% by Week 8. Reduction in proteinuria 25% at Week 8 was predictive of renal response at Week 24. The odds ratio (95% Confidence Intervals) of renal response at Week 24 for those subjects with ≥25% reduction in proteinuria at Week 8 compared to those with <25% reduction in proteinuria at Week 8 was 3.2 (2.1-5.1) (Dall'Era M, Stone D, Levesque V, Cistemas M, Wofsy D. Identification of biomarkers that predict response to treatment of lupus nephritis with mycophenolate mofetil or pulse cyclophosphamide. Arthritis care & research. 2011:63(3):351-7).

Administration

The diluted solution of VIB4920 will be administered intravenously via infusion pump over approximately 90 minutes. The infusion may be slowed or interrupted if the subject develops an infusion reaction. VIB4920 will be administered by trained medical personnel prepared to manage anaphylaxis, severe hypersensitivity reactions, and infusion-related reactions. VIB4920 should be discontinued immediately for serious hypersensitivity reactions or for severe and life-threatening infusion reactions.

Monitoring

Vital signs must be assessed prior to each infusion and assessed approximately every 30 minutes during the infusions. After completion of each infusion the IV line should remain in the subject for at least 1 hour.

The subject's vital signs must be assessed approximately every 30 minutes for 2 hours after the completion of the first 3 infusions, and for 1 hour after completion of the remaining infusions.

Placebo

Placebo is 0.9% normal saline. Placebo will be administered intravenously at Weeks 10, 12, 14, 18, 22, 26, 30, and 34, according to schedules provided herein. Placebo will be a 250 mL bag of 0.9% normal saline. The infusion bag should be masked. Placebo will be administered according to the instructions provided for VIB4920.

Primary Objective

Evaluate the efficacy of VIB4920 antagonism of CD40L in achieving a complete renal response in active LN. Secondary Objective is to evaluate the efficacy of VIB4920 antagonism of CD40L in active LN: (1) Achieving a renal response; (2) Reducing anti-dsDNA antibodies; (3) Reducing hypocomplementemia; (4) Reducing SLE disease activity and damage accrual; and/or (5) Preventing renal treatment failure events. Secondary safety objectives are to evaluate the safety of VIB4920 antagonism of CD40L in active LN: (1) Serious adverse events; (2) Adverse events of special interest; and/or (3) Serum immunoglobulin levels.

Primary Endpoint

The primary endpoint, complete renal response, will be assessed at Week 38. Primary Endpoint is complete renal response at Week 38, defined as all of the following: (1) UPCR ≤0.75, based on a 24-hour collection; (2) Estimated glomerular filtration rate (eGFR) ≥120 ml/min/1.73 m2, or if <120 ml/min/1.73 m2, then >80% of the eGFR at baseline; (3) Prednisone ≤5 mg/day from Week 8, according to the prednisone dosing restrictions specified herein.

Secondary efficacy endpoints comprise: (1) Complete renal response at Weeks 26, 48, and 60; and as defined herein; (2) Overall renal response at Weeks 26, 38, 48, and 60, defined as all of the following: (a) ≥50% improvement in the UPCR compared to baseline, based on a 24-hour urine collection; (b) Estimated eGFR ≥120 ml/min/1.73 m2, or if <120 ml/min/1.73 m2, then 80% of the eGFR at baseline; and (c) Prednisone ≤5 mg/day from Week 8, according to the prednisone dosing restrictions described herein: (3) UPCR at Weeks 26, 38, 48, and 60, based on a 24-hour urine collection; (4) Anti-dsDNA antibodies at Weeks 26, 38, 48, and 60; (5) C3 levels at Weeks 26, 38, 48, and 60; (6) C4 levels at Weeks 26, 38, 48, and 60; (7) SLEDAI-2K at Weeks 26, 38, 48 and 60; (8) SLICC/ACR-DI at Weeks 26 and 60; and/or (9) Renal treatment failures as defined herein.

Secondary safety endpoints comprise: (1) Serious adverse events; (2) adverse events such as: (a) Anaphylaxis; (b) Grade 3 or greater infusion reaction; (c) Grade 3 or greater hypersensitivity reaction; (d) Grade 3 or greater infection; and/or (e) Thromboembolic event; (3) Serum IgM and IgG levels at Weeks 26, 38, 48, and 60.

Complete renal response will be defined as a composite of three criteria. The rationale for each component of the primary endpoint for the current trial is the following:

1. UPCR ≤0.75, based on a 24-hour collection. The cut-off of UPCR ≤0.75 was selected based on evidence that UPCR in the range of 0.7 to 0.8 is a predictor of good long term renal outcomes in LN (Dall'Era M, Cistemas M G, Smilek D E, Straub L, Houssiau F A, Cervera R, et al. Predictors of long-term renal outcome in lupus nephritis trials; lessons learned from the Euro-Lupus Nephritis cohort. Arthritis Rheumatol. 2015; 67(5):1305-13; Tamirou F, Lauwerys B R, Dall'Era M, Mackay M, Rovin B, Cervera R, et al. A proteinuria cut-off level of 0.7 g/day after 12 months of treatment best predicts long-term renal outcome in lupus nephritis; data from the MAINTAIN Nephritis Trial. Lupus Sci Med. 2015; 2(1):e000123. In the BLISS-LN trial, UPCR ≤0.7 was a component of the primary efficacy renal response definition (Furie R, Rovin B H, Houssiau F, Malvar A, Teng Y K O, Contreras G, et al. Two-Year, Randomized, Controlled Trial of Belimumab in Lupus Nephritis. The New England journal of medicine. 2020; 383(12):1117-28).

2. Estimated glomerular filtration rate (eGFR) 120 ml/min/1.73 m² or, if <120 m./min/1.73 m², then >80% of the eGFR at baseline. The CKD-EPI formula will be utilized to calculate eGFR (Levey A S, Stevens L A, Schmid C H, Zhang Y L, Castro A F, 3rd, Feldman H I, et al. A new equation to estimate glomerular filtration rate. Annals of internal medicine. 2009:150(9):604-12). eGFR ≥120 ml/min/1.73 m² is included because the percent change at high values of eGFR may be misleading and result in misclassifying subjects as not having a renal response.

3. Prednisone ≤5 mg/day from Week 8, according to the prednisone dosing restrictions specified herein.

Exploratory Endpoints

Exploratory efficacy endpoints comprise change in histologic activity on the Week 38 renal biopsy.

Exploratory investigational Agent endpoints comprise: (1) Plasma concentrations of VIB4920 and non-compartmental pharmacokinetic (PK) parameters, and (2) ADA to VIB4920. The association between pharmacokinetic parameters and baseline disease characteristics and clinical outcomes including proteinuria will be explored using longitudinal models for repeated measures.

Additional Study Details

Accrual Objective: 66 randomized subjects. Up to 114 subjects may be enrolled to randomize 66, depending on the actual randomization rate.

Study Duration: 50 months: Approximately 36 months enrollment+14 months subject study participation.

Treatment description: VIB4920 1500 mg or placebo intravenously at Weeks 10, 12, 14, 18, 22, 26, 30, and 34, while continuing on MMF and prednisone.

Timing of Analysis. Since the primary endpoint occurs prior to the completion of the study, analysis of the primary and secondary endpoints through Week 38 will be conducted when active study subjects have completed the Week 38 assessments, and data collected through Week 38 is frozen. Analysis of the other secondary endpoints will be conducted after subjects have completed study participation through Week 60 and all data collected is locked.

Primary analysis of primary endpoint. The primary endpoint is the proportion of subjects achieving a complete renal response at Week 38, and will be calculated as a binary response (Yes or No) indicating whether the subject met the criteria for complete renal response at Week 38, per the primary endpoint definition provided herein. The primary analysis of the primary endpoint will be performed on the mITT sample and is designed to test the following hypothesis:

Null hypothesis: The proportion of subjects achieving a complete renal response at Week 38 does not differ between the VIB4920 and placebo arms.

Alternative hypothesis. The proportion of subjects achieving a complete renal response at Week 38 is greater in the VIB4920 arm compared to the placebo arm.

An exact logistic regression analysis will be used to perform the one-sided test to determine if the proportion of subjects who achieved a complete renal response was higher in the VIB4920 arm compared to the placebo arm. The exact logistic regression model will use the binary response variable for complete renal response status as the dependent variable, the treatment arm as the independent variable, and the three-level stratification factor defined herein as a covariate. The test comparing treatment arms will be evaluated using a Type 1 one-sided error rate of α=0.10. The primary estimand is defined to be the difference in the proportion of subjects with lupus nephritis in the VIB4920 arm versus the placebo arm who achieve a complete renal response at Week 38 without discontinuing treatment early due to worsening lupus nephritis. The target population for the estimand will be the subjects who meet the criteria for the mITT analysis population. Intercurrent events will be analyzed by methods to best inform the estimand. A composite variable strategy will be applied to the intercurrent event in which subjects who discontinue study treatment for reasons provided herein prior to Week 38, such that they will be analyzed as not achieving a complete renal response in the primary estimand. The treatment policy strategy will be applied to subjects who discontinue for reasons described herein prior to Week 38, since these subjects will continue to be followed for renal response, and will be analyzed at Week 38 according to the complete renal response criteria. The treatment policy strategy will also be followed for subjects with dose modification of MMF and prednisone as outlined herein, and the subjects will be analyzed at Week 38 according to the complete renal response criteria. Missing data at Week 38 for the variables to calculate the primary endpoint will be imputed according to the most appropriate imputation method. Subjects with a missing 24-hour UPCR result at Week 38 will be imputed using the subjects' spot UPCR result at Week 38. Subjects for whom the response criteria cannot be assessed at Week 38 due to missing information for either UPCR or eGFR will have their complete renal response status imputed by taking the last observation carried forward (LOCF) from the Week 34 assessment. Subjects who have not met the criteria for the intercurrent event of discontinuation for reasons described herein, but are missing both Week 34 and 38 assessments, will be imputed via multiple imputation according to specifications in the statistical analysis plan.

Supportive Analyses of the Primary Endpoints. The primary analysis of the primary endpoint will be repeated in subjects who meet the criteria for the mITT analysis population with the following assumptions to assess the robustness of the endpoint definition and primary estimand. Sensitivity analyses will be conducted using a 24-hour UPCR ≤0.5 as the UPCR criterion for complete response, along with additional analyses using inactive urinary sediment as an additional criterion for complete response. Inactive urinary sediment will be defined as meeting all of the following criteria: Urinary RBC reported in a range of less than 5-10/hpf, in the absence of menses and infection, and Urinary WBC reported in a range of less than 5-10/hpf, in the absence of infection, and Absence of RBC and WBC casts. Additional sensitivity analyses will use a composite variable strategy of defining all subjects with missing Week 38 estimate as not achieving a complete response and using a complete case analysis. The primary estimator of the primary endpoint will be repeated on the PP sample. Additional sensitivity analyses will be performed on the subset of subjects in the PP sample who completed the Week 38 visit, received 7 of the 8 doses of VIB4920 or placebo, and had no major protocol deviations that impacted efficacy assessments.

Analyses of Secondary Efficacy Endpoints. The secondary efficacy endpoints, the endpoint definitions, and time points when the endpoints will be analyzed are provided herein. All secondary inferential analyses are considered supportive; p-values for tests of differences between groups will be presented without adjustment for multiple comparisons. The mITT and PP samples will be used for all secondary analyses. For the renal response endpoints, intercurrent events will be analyzed by methods to best inform the estimand. A composite variable strategy will be applied to the intercurrent event in which subjects who discontinue study treatment for the reasons provided herein prior to the endpoint, such that they will be analyzed as not achieving a renal response in the estimand. The treatment policy strategy will be applied to subjects who discontinue for reasons provided herein prior to the endpoint, since these subjects will continue to be followed for renal response and will be analyzed at the endpoint according to the renal response criteria. The treatment policy strategy will also be followed for subjects with dose modification of MMF and prednisone as outlined herein, and the subjects will be analyzed at the endpoint according to the renal response criteria. Missing data at the endpoint for the variables to calculate the renal response will be imputed according to the most appropriate imputation method. Subjects with a missing 24-hour UPCR result at the time point will be imputed using the subjects' spot UPCR result at that time point. Subjects for whom the renal response criteria cannot be assessed at the endpoint due to missing information for either UPCR or eGFR will have their renal response status imputed by taking the LOCF from the most recent assessment, provided the most recent assessment is within 4 weeks of the endpoint being imputed. Subjects who have not met the criteria for the intercurrent event of discontinuation for reasons provided herein and for whom the renal response criteria cannot be assessed at the endpoint due to missing information for either UPCR or eGFR and do not have an assessment within 4 weeks of the endpoint will be excluded from analysis. The null hypothesis proposes that there are no differences in the secondary endpoints (measured either as means or proportions) between study groups. The alternative hypothesis proposes that there are differences between groups.

The following analyses are planned to evaluate the impact of the study treatments over the course of the study: (1) The proportions of subjects who met the criteria for complete renal response and overall renal response will be analyzed using an exact logistic regression model with the binary response variable for renal response status at the time point as the dependent variable, the treatment arm as the independent variable, and the three-level stratification factor as a covariate. Exploratory analyses will also consider the proportion of subjects who met the criteria for overall renal response when the assessments of UPCR and eGFR at the Week 8 visit are used instead of Visit −1 to define the reference point for changes. The overall renal response definition for changes from Week 8 will be defined as all of the following: (a) ≥50% improvement in the UPCR compared to Week 8, or UPCR ≤0.75, based on a 24-hour urine collection, and (b) eGFR ≥120 ml/min/1.73 m2, or if ≤120 ml/min/1.73 m2, then ≥80% of the eGFR at Week 8, and (c) Prednisone ≤5 mg/day from Week 8, according to the prednisone dosing restrictions specified herein.

For the secondary endpoint of UPCR levels at Week 38 as a continuous measure, the absolute value and percent change from baseline will be summarized in each arm using descriptive statistics, including the median and bootstrapped BCa confidence interval. A rank-based Wilcoxon-Mann-Whitney (WMW) test will be used to evaluate if UPCR levels are lower in the VIB4920 arm compared to the placebo arm. Subjects that fail the primary endpoint for excessive estimated eGFR or prednisone usage will be imputed as the lowest rank for the WMW test. Supportive analyses will conduct a van Elteren test with the stratification factor defined herein as a covariate. Exploratory analyses will consist of summaries by treatment arm of the descriptive statistics for the percent change from Week 8. The change in the proportion of subjects who had a negative anti-dsDNA test after initiation of VIB4920 or placebo will be summarized by arm, and will be analyzed using an exact conditional logistic regression model with the binary response variable for anti-dsDNA status at the time point as the dependent variable, the treatment arm as the independent variable, and the anti-dsDNA status at Week 10 as the covariate. Exploratory analyses will also consider within-subject changes in anti-dsDNA status from baseline using exact methods for binary variables. The change in the proportion of subjects who were hypocomplementemic for C3 and C4 after initiation of VIB4920 or placebo will be summarized by arm, and analyzed using an exact conditional logistic regression model with the binary response variable for the test result (C3 or C4) status at the time point as the dependent variable, the treatment arm as the independent variable, and the test result at Week 10 as the covariate. Exploratory analyses will also consider within-subjects changes in C3 and C4 status from baseline using exact methods for binary variables. The change in SLEDAI-2K scores after initiation of VIB4920 or placebo will be summarized by arm, and analyzed using an analysis of covariance (ANCOVA) model with the SLEDAI-2K score at the timepoint as the dependent variable, the treatment arm as the independent variable, and the SLEDAI-2K score at Week 0 as the covariate. Exploratory analyses for the change in SLEDAI-2K score from randomization will be analyzed using an ANCOVA model with the SLEDAI-2K score at the time point as the dependent variable, the treatment arm as the independent variable, and the SLEDAI-2K score at Week 10 as the covariate. If the validity of the normality assumption is in question, the WMW tests will also be performed. The change in SLICC/ACR-DI scores after initiation of VIB4920 or placebo will be summarized by arm, and analyzed using an ANCOVA model with the SLICC/ACR-DI score at the time point as the dependent variable, the treatment arm as the independent variable, and the SLICC/ACR-DI score at Week 0 as the covariate. If the validity of the normality assumption is in question, the WMW tests will also be performed.

Study Population

LN is a common and serious manifestation of SLE, with a high risk of progressing to end stage renal disease. Therapies available for LN have incomplete efficacy and high toxicity. The eligible study population will be subjects with active LN who are appropriate for a clinically indicated renal biopsy and treatment with MMF. Subjects must have a UPCR of 1.5 or higher, since the primary endpoint of complete renal response requires UPCR ≤0.75, and at least 50% improvement in proteinuria should be required in order to meet the primary endpoint. They may not have active or latent infections, since infection is a potential risk of the investigational agent. The risk of fetal harm with VIB4920 is unknown, and MMF is known to cause fetal harm. Therefore, subjects may not be pregnant, breast-feeding, or unwilling to use contraception if female of child-bearing potential or if male with a partner of child-bearing potential.

Inclusion Criteria: Subjects who meet all of the following criteria are eligible for enrollment as study subjects: (1) Age 18 years or older: (2) Classification of Systemic Lupus Erythematosus (SLE) by any of the following criteria: the 1997 update of the 1982 American College of Rheumatology (ACR) criteria, the 2012 Systemic Lupus International Collaborating Clinics (SLICC) criteria, or the 2019 European League Against Rheumatism (EULAR)/ACR criteria; (3) UPCR 1.5 based on a 24-hour urine collection at Visit −1 or within 14 days prior to Visit −1; and (4) Renal biopsy documentation at Visit −1 of ISN/RPS LN: Class III, Class IV, or Class V in combination with Class III or IV, and modified NIH activity index ≥1.

Exclusion Criteria: Subjects who meet any of these criteria are not eligible for enrollment as study subjects:
1. Inability or unwillingness to give written informed consent or comply with study protocol.
2. Contraindication to treatment with MMF or mycophenolate sodium: or treatment with MMF or mycophnolate sodium is inappropriate in the opinion of the investigator.
3. Treatment with a biologic agent or investigational agent within 90 days or 5 half-lives prior to Visit 0, whichever is longer.
4. Rituximab or other B cell depleting agent within 6 months prior to Visit 0.
5. Prior treatment with VIB4920.
6. Receipt of a live attenuated vaccine within 4 weeks prior to Visit 0.
7. Comorbidities requiring treatment with systemic corticosteroids, including those that have required 3 or more courses of systemic corticosteroids within 12 months prior to Visit 0.
8. Current malignancy or history of malignancy, except for adequately treated basal cell carcinoma, squamous cell carcinoma, or cervical carcinoma in situ >12 months prior to Visit 0.
9. End stage renal disease, defined as eGFR <20 ml/min/1.73 m².
10. History of transplantation.
11. The following risks for thromboembolic events:
   a. Recent or recurrent deep venous thrombosis or arterial thromboembolism.
   b. Immobilization or major surgery within 12 weeks prior to Visit 0.
   c. History of congenital or inherited deficiency of antithrombin III, protein S, or protein C.
   d. History of anti-phospholipid syndrome.
   e. Any one of the following anti-phospholipid antibodies:
      i. Positive lupus anticoagulant test, or
      ii. Anti-β₂-glycoprotein I IgG ELISA titer ≥40 GPL, or
      iii. Anti-cardiolipin IgG ELISA titer ≥40 GPL.
12. History of a severe allergy or hypersensitivity reaction to any component of the VIB4920 formulation.
13. Any one of the following laboratory abnormalities:
   a. Peripheral B cell count <5/μl.
   b. Neutropenia (absolute neutrophil count <1000/mm³).
   c. Anemia (hemoglobin <8 g/dL).
   d. Thrombocytopenia(platelets <50,000/mm³).
   e. Aspartate aminotransferase or alanine aminotransferase ≥2× upper limit of normal.
14. Evidence of current or prior tuberculosis infection, including any of the following:
   a. Positive QuantiFERON-TB Gold or TB Gold Plus test.
   b. Positive T-SPOT.TB test.
   c. Positive purified protein derivation (PPD) tuberculin, defined as >5 mm induration.
15. Human immunodeficiency virus (HIV) infection.
16. Current or past hepatitis B (HBV) infection.
17. Current or past hepatitis C virus (HCV) infection, except adequately treated HCV with documented sustained virologic response.
18. Active bacterial, viral, fungal, or opportunistic infection.
19. History of significant, recurrent, or chronic infection that may pose additional risks from participating in the study, in the opinion of the investigator.
20. History of severe psychiatric condition that would interfere with the subject's ability to comply with the study protocol, in the opinion of the investigator.
21. Current substance abuse, or history of substance abuse within 12 months prior to Visit 0.
22. Lack of peripheral venous access.
23. Pregnancy.
24. Breastfeeding.
25. Unwillingness to use a medically acceptable form of contraception for the duration of the study if female of child-bearing potential or if male with a partner of child-bearing potential.
26. Past or current medical problems or findings from physical examination or laboratory testing that are not listed above, which, in the opinion of the investigator, may pose additional risks from participation in the study, may interfere with the subject's ability to comply with study requirements or that may impact the quality or interpretation of the data obtained from the study.

In addition to the pre-scheduled data reviews and planned safety monitoring, the DSMB may be called upon for ad hoc reviews. The DSMB will review any event that potentially impacts safety at the request of the protocol chair or DAIT/NIAID. In addition, the following events occurring in randomized subjects will trigger an ad hoc comprehensive DSMB Safety Review: (1) Any death that is at least possibly related to the investigational agent; (2) Two Grade 4 infectious AEs involving two different subjects; (3) Two Grade 2 or greater thromboembolic events involving two different subjects; and/or (4) Two life-threatening infusion reactions, hypersensitivity reactions, or anaphylaxis events that lead to premature discontinuation of the investigational agent. The DSMB will review the safety data within two weeks of the notification to the sponsor of the event. If the DSMB has not reviewed the safety data within two weeks, study enrollment will be suspended and no new subjects will sign informed consent. Subjects already in screening may continue with screening assessments, and subjects already enrolled will continue as planned pending DSMB review. After review of the data, the DSMB will make recommendations regarding study conduct and/or continuation.

Renal Treatment Failure

Renal treatment failure is characterized as any one of the following.
1. Worsening proteinuria, defined as both of the following.
a. UPCR ≥1.5, and
b. ≥50% increase in UPCR compared to the lowest previous value. Two successive evaluations at least one week apart must be performed. The first evaluation may be based on either a spot urine or a 24-hour urine collection. The confirmatory evaluation must be based on a 24-hour urine collection.
2. Progressive deterioration in renal function, defined as both of the following:
a. Serum creatinine ≥1.5, and
b. ≥50% increase in serum creatinine compared to the lowest previous value. Two successive evaluations at least one week apart must be performed.
3. Other renal treatment failure events may occur during the trial, including investigator decision to discontinue the subject from study therapy due to nephritis that worsens or fails to improve sufficiently, or receipt of a prohibited immunosuppressive or immunomodulatory medication for treatment of LN. These events will be evaluated in blinded fashion by a renal treatment failure event adjudication committee consisting of the protocol chair, the DAIT/NIAID medical monitor, and the ITN clinical trials physician.

Randomization

At Week 10, subjects who meet all of the following criteria will be randomly assigned 2:1 in favor of treatment with VIB4920 versus treatment with placebo.
1. Week 8 UPCR >0.75, based on a 24 hour urine collection.
2. MMF dose is 2-3 g/day by Week 4, and maintained.
3. Prednisone dose is 5 mg/day by Week 8, and maintained.

Random assignment will be stratified by proteinuria levels at baseline and at Week 8 as follows:

UPCR <3 at baseline

UPCR ≥3 at baseline that improves by ≥25% at Week 8

UPCR ≥3 at baseline that improves by <25% at Week 8

The randomization will be performed by the Division of Allergy, Immunology, and Transplantation Statistical and Clinical Coordinating Center (DAIT-SACCC).

Toxicity Prevention and Management

VIB4920 or placebo must be suspended if the subject develops a Grade 3 or greater infection. VIB4920 or placebo also must be suspended for symptomatic or asymptomatic SARS-CoV-2 infection.

VIB4920 or placebo may also be suspended if the subject develops any AE that the investigator judges to be significant.

If the infection or the AE resolves. VIB4920 or placebo may be restarted at the next scheduled dose.

VIB4920 or placebo will be permanently discontinued in a subject for any of the following adverse events.
a. Anaphylaxis, defined according to definitions provided herein.
b. Grade 3 or greater infusion reaction
c. Grade 3 or greater hypersensitivity reaction
d. Thromboembolic event
e. Grade 4 infection
f. Grade 3 or greater opportunistic infection
g. Malignancy
h. Hepatic function abnormality that meets the following criteria: an increase in ALT or AST >3 times the upper limit of normal (ULN) and concurrent increase in bilirubin >2 times ULN. If VIB4920 or placebo is permanently discontinued for any of the events listed above, the procedures provided below will be followed.

Risks and Benefits to Subjects

Anaphylaxis, Serious Allergic Reactions, and Infusion-Related Reactions.

VIB4920 is an engineered foreign protein, and, as such, may induce an acute reaction following infusion, including IgE mediated anaphylactic or hypersensitivity reactions and anaphylactoid reactions. The reactions may be severe or fatal, and may include cardio-respiratory, skin, and gastrointestinal signs and symptoms, such as chest pain, hypotension, dyspnea, bronchospasm, respiratory failure, urticaria, pruritus angioedema, nausea, vomiting, diarrhea, hyptonia, and collapse. Infusion reactions may include local reaction at the infusion site, pyrexia, urticaria, arthralgia, bronchospasm, wheeze, cough, dizziness, dyspnea, fatigue, headache, hypertension, hypotension, myalgia, and vomiting.

Subjects with a history of severe allergy or reaction to any component of the VIB4920 formulation may not be eligible to participate in the study. VIB4920 will be administered by trained medical personnel prepared to manage anaphylaxis, severe hypersensitivity reactions, and infusion-related reactions. Vital signs will be monitored during the infusion, and the subject will be observed at the clinical site for one hour following the infusion.

Immune Complex Disease

Immune complex disease is a potential risk of VIB4920 administration due to the potential to generate ADA. As noted herein, the impact of ADA on the pharmacokinetics of VIB4920 has only been observed at doses ≤100 mg. Immune complex disease manifestations could include arthralgias, serum-sickness, nephritis, and vasculitis.

Infections

The CD40:CD40L costimulatory pathway is central to the development of a comprehensive immune response, including the host immune response to pathogens. Interference with this costimulatory pathway therefore may increase the risk of infection. In pre-clinical studies, one cynomolgous monkey developed an opportunistic fungal infection, while a second monkey developed signs consistent with systemic infection as described herein. In humans, there were several non-serious herpes AEs. There was also one SAE of Grade 4 encephalitis of uncertain etiology, deemed unrelated to VIB4920 by the investigator.

Subjects will not be eligible to participate if they have an active bacterial, viral, fungal, or opportunistic infection, or if they have a current or recent infection that may pose additional risks. Subjects with evidence of current or past tuberculosis, human immunodeficiency virus (HIV), hepatitis B virus (HBV), or hepatitis C virus (HCV) will not be eligible to participate, except for adequately treated HCV with documented sustained virologic response.

It is recommended that subjects are up to date on standard vaccinations prior to study participation. Receipt of a live attenuated vaccine will not be permitted within 4 weeks of enrollment into the study, nor during treatment with VIB4920, nor for 12 weeks after VIB4920 is discontinued.

There is risk of infection during trial participation, including risk of SARS-CoV-2 and other types of infections. Subjects will be educated and advised about the importance of strict adherence to the Center for Disease Control and Prevention (CDC) recommendations for reducing the risk of SARS-CoV-2 infection, as well as the risk that immune response to SARS-CoV-2 vaccination may be suboptimal due to study treatment. SARS-CoV-2 vaccination is permitted during the study, however it is strongly recommended that vaccination be completed prior to receipt of study medication whenever possible. During study conduct, VIB4920 will be suspended if the subject develops a Grade 3 or greater infection. VIB4920 also will be suspended if the subject develops symptomatic or asymptomatic SARS-CoV-2 infection.

VIB4920 will be permanently discontinued if the subject develops a Grade 4 infection or a Grade 3 or greater opportunistic infection.

Thromboembolism

Venous and arterial thromboembolic AEs occurring with antibodies targeting CD40L led to discontinuation of drug development programs for these agents. Subsequent non-clinical experiments identified platelet activation due to binding of the Fc receptor by immune complexes of CD40L and anti-CD40 antibodies as the likely cause of thromboembolism. VIB4920 is an engineered protein that lacks an Fc domain and does not promote platelet aggregation or thromboembolic effects in preclinical studies. No thromboembolic events have occurred with VIB4920 in animals or humans.

Thromboembolism is a known risk of LN, especially in subjects with nephrotic range proteinuria VIB4920 is not expected to increase this pre-existing risk. As an added safety measure, subjects with several risk factors for thromboembolic events will be excluded from the trial including recent or recurrent deep venous thrombosis or arterial thromboembolism, recent immobilization or major surgery, history of congenital or inherited deficiencies in antithrombin III, protein S, and protein C, history of anti-phospholipid syndrome, and a high-risk anti-phospholipid antibody profile.

Exposure in Utero

Embryo-fetal studies have not been conducted. Pregnant and breast-feeding subjects will not be eligible to participate in the trial, and female subjects of child-bearing potential will be required to use at least one method of highly effective contraception. Male subjects with a female partner of child-bearing potential will also be required to use contraception.

Concomitant Medications

Subjects should be enrolled in the Mycophenolate Risk Evaluation and Mitigation Strategy (REMS) program.

All subjects will receive mycophenolate mofetil (MMF) beginning at Day 0. The maximum tolerated dose of 2 to 3 g/d must be reached by Week 4. MMF will be maintained at the maximum tolerated dose through Week 60, except as specified herein. After Week 38, a dose of 1 to 1.5 g/d is permitted due to clinical intolerance. Subjects with eGFR <25 mL/min/1.73 m² will receive a maximum MMF dose of 1 g twice a day. Mycophenolate sodium may be substituted for MMF at an equivalent dose.

MMF may be suspended for a maximum of 7 days, or the dose may be reduced to ≥1 g/d for a maximum of 14 days, for the following reasons. (1) Clinical intolerance; (2) Leukopenia 2000/mm3: and/or (3) Neutropenia ≤1500/mm3. Following suspension or dose reduction, the previous maximum tolerated dose of 2 to 3 g/d must be reached within ≤4 weeks. MMF will be permanently discontinued if suspension or dose reduction less than the target dose is required a second time.

Prednisone. Subjects will receive prednisone 25 mg/d beginning Day 0, or on the day after completion of methylprednisolone. Prednisone will be tapered to 5 mg/d beginning at Week 8, according to Table 2. Prednisone will be continued at 5 mg/d until Week 60. Prednisone administration will be in accordance with the prescribing information.

TABLE 2
Exemplary Prednisone Tapering Schedule

| Study Week | Dose of Prednisone (mg/day) |
|---|---|
| 0-2 | 25 |
| 2 | 20 |
| 4 | 15 |
| 6 | 10 |
| 8 | 5 |

Following randomization, prednisone may be increased once during the study for non-renal SLE disease activity and once for conditions unrelated to SLE, at the discretion of the site investigator. The dose may be increased for a period of ≤14 days, not to exceed 25 mg/d. An equivalent dose of another corticosteroid may be substituted for prednisone. The prednisone dose may not exceed 5 mg/d for >14 days. The prednisone dose may not exceed 5 mg/d between Week 34 and Week 38. Dose modification of prednisone is not permitted prior to randomization.

Methylprednisolone. Subjects will receive a total of 1000 mg of methylprednisolone according to either of the following schedules: 1000 mg methylprednisolone IV at Day 0, or 500 mg methylprednisolone IV at Day 0 and at Day 1. Subjects who previously received 1000 mg of methylprednisolone IV at Visit −1 or within 14 days prior to Visit −1 will not receive additional methylprednisolone IV on Day 0. Subjects who previously received less than 1000 mg of methylprednisolone IV at Visit −1 or within 14 days prior to Visit −1 will receive an additional dose of methylprednisolone IV at Day 0, according to the following formula, where X is the intravenous dose previously received and Y is the intravenous dose administered on Day 0: 1000 mg−X=Y. Methylprednisolone administration will be in accordance with the prescribing information.

Nonsteroidal Anti-inflammatory Drugs. Initiation of nonsteroidal anti-inflammatory drugs (NSAIDs) during the trial is not recommended due to the possible adverse effect on renal function. However, NSAIDs are permitted if necessary, for control of symptoms.

Treatment of Hypogammaglobulinemia. Treatment of hypogammaglobulinemia in subjects with infectious AEs is permitted at the discretion of the site investigator, in consultation with the protocol chair and the DAIT/NIAID medical monitor.

SARS-CoV-2 Vaccines. SARS-CoV-2 vaccines are permitted if they have FDA emergency use authorization or are FDA-approved. It is strongly recommended that vaccination be completed prior to the first dose of study medication.

Prophylactic Medications

Osteoporosis Treatment and Prevention. Measures to prevent and to treat osteoporosis are strongly encouraged during this trial. These measures may include any of all of the following: Calcium carbonate or citrate (1500 mg/d); Vitamin D (up to 2000 IU/d); and/or Bisphosphonates.

Cholesterol Control. At the discretion of the site investigator, subjects may be treated with a cholesterol-lowering agent such as a statin.

Antimalarials. Treatment with an antimalarial agent such as hydroxychloroquine is encouraged unless contraindicated.

Blood Pressure Control. All subjects not already on either an angiotensin converting enzyme inhibitor (ACEi) or an angiotensin receptor blocker (ARB) may be started on such an agent unless contraindicated. The dose should be adjusted to achieve a targeted systolic blood pressure less than 130 mmHg. A combination of medications may be used if a single agent does not control systolic blood pressure, including an ACEi, an ARB, a calcium channel blocker, and/or a beta blocker.

In aspects, none of the aforementioned prophylactic medications are required.

Prohibited Medications

Immunosuppressive and Immunomodulatory Medications. Immunosuppressive and immunomodulatory medications are prohibited except for the protocol mandated medications specified herein. Prohibited immunosuppressive medications include but are not limited to cyclophosphamide, azathioprine, rituximab, ocrelizumab, belimumab, eculizumab, abatacept, plasmapheresis/plasma exchange, and calcineurin inhibitors. Intravenous immunoglobulin is also prohibited except for treatment of hypogammaglobulinemia with an infectious AE.

Oral and intravenous corticosteroids are prohibited except, or if required to treat anaphylaxis, hypersensitivity, or an infusion reaction.

Investigational agents or treatments are prohibited during study participation.

Live-attenuated vaccines are prohibited during treatment with the investigational agent, and for 12 weeks after the investigational agent is discontinued.

Rescue Medications

Rescue medications are permitted for treatment of anaphylaxis, hypersensitivity, and infusion reactions, including but not limited to epinephrine, corticosteroids, diphenhydramine, acetaminophen, and bronchodilators. A temporary increase in prednisone dose for non-renal SLE disease activity is permitted. Rescue medications for renal treatment failure as described herein, worsening lupus nephritis and renal flare are not permitted. Subjects requiring additional immunomodulatory or immunosuppressive medications will be discontinued from study medication as described herein.

Contraception

All female subjects of childbearing potential must not become pregnant during the study and must either be sexually inactive by abstinence or use at least one highly effective, medically acceptable form of contraception for the duration of their study participation in the trial. Periodic abstinence and withdrawal are not acceptable methods of contraception. Medically acceptable forms of birth control include injectable or implantable progestogens, intrauterine devices, estrogen vaginal rings, bilateral tubal occlusion, or male partner sterilization. Oral contraceptive pills are permitted as a contraceptive method in combination with a barrier method, according to MMF REMS guidance (Mycophenolate REMS. 2020). If using male partner sterilization (vasectomy with documentation of azoospermia), sterilization must occur prior to the female subject's entry into the study, and the male must be the sole partner for the subject.

Non-sterilized male subjects with a female partner of childbearing potential are required to use a condom with spermicide. It is recommended that female partners of childbearing potential also use a highly effective method of birth control as described above.

Study Assessments

The study assessments to be performed at each study visit are listed in Table 5 and Table 6.

In brief, general assessments comprise: Informed consent: Written informed consent will be obtained before any study assessments or procedures are performed: Eligibility criteria: Eligibility for study participation will be assessed during the screening period; Demographics: age, gender and ethnicity; Medical history; A history will be taken to determine if the subject has had any clinically significant diseases or medical procedures other than the disease under study; Vital signs: Height will be obtained at Visit −1; weight, temperature, pulse, blood pressure, and respiration will be obtained at all visits: Comprehensive physical examination including the following body systems: skin, respiratory, cardiovascular, gastrointestinal, neurologic, musculoskeletal, and head, ears, eyes, nose, and throat (HEENT). Limited physical examination including all body systems relevant to the subject's clinical complaints and clinical status at the study visit; Lupus history to determine date of diagnosis, onset of lupus nephritis, and onset of the current lupus nephritis flare.

Adverse events. Subjects will be assessed for AEs: Concomitant medications. All concomitant medications and their indications will be recorded.

Disease-specific assessments comprise: Systemic Lupus Erythematosus Disease Activity Index 2000 (SLEDAI-2K); and/or Systemic Lupus International Collaborating Clinics/American College of Rheumatology Damage Index for Systemic Lupus Erythematosus (SLICC/ACR-DI).

Clinical Laboratory Assessments comprise: Hematology: CBC, differential and platelet count; Chemistry: AST, ALT, bilirubin, alkaline phosphatase, albumin, creatinine, eGFR calculated according to the CKD-EPI formula (Levey A S, Stevens L A, Schmid C H, Zhang Y L, Castro A F, 3rd, Feldman H I, et al. A new equation to estimate glomerular filtration rate. Annals of internal medicine. 2009:150(9):604-12); CD19; HIV. Unless test has been performed within 30 days of Visit −1 and documented test results are available; Hepatitis B: surface antibody, core antibody, and surface antigen. Unless test has been performed within 30 days of Visit −1 and documented test results are available: Hepatitis C: RNA or antibody. Unless test has been performed within 30 days of Visit −1 and documented test results are available: Tuberculosis Testing: QuantiFERON—TB Gold or TB Gold Plus. Unless test has been performed within 30 days of Visit −1. T-SPOT.TB test or PPD tuberculin test may substitute. In the case of an indeterminate QuantiFERON-TB Gold or TB Gold Plus test, a PPD tuberculin test may substitute; Serum pregnancy; Urine pregnancy; Spot Urine: protein, creatinine, protein-to-creatinine ratio, albumin-to-creatinine ratio: Urinalysis; 24-Hour Urine: protein, creatinine, protein-to-creatinine ratio; Serum Immunoglobulins: IgG, IgA, IgM; Anti-phospholipid Antibodies: lupus anticoagulant, anti-glycoprotein I IgG, anti-cardiolipin IgG; Anti-dsDNA; C3, C4; and/or Renal biopsy histology; ISN/RPS classification (Bajema I M, Wilhelmus S, Alpers C E, Bruijn J A, Colvin R B, Cook H T, et al. Revision of the International Society of Nephrology/Renal Pathology Society classification for lupus nephritis: clarification of definitions, and modified National Institutes of Health activity and chronicity indices. Kidney international. 2018:93(4):789-96; Weening J J, D'Agati V D, Schwartz M M, Seshan S V, Alpers C E, Appel G B, et al. The classification of glomerulonephritis in systemic lupus erythematosus revisited. Kidney international. 2004; 65(2):521-30), activity index, chronicity index, and assessment of vascular lesions including thrombotic microangiopathy.

VIB4920 Assessments comprise: Plasma PK assays, Urine PK assays, Plasma ADA assays, Plasma sCD40L assays, and combination thereof.

Mechanistic Assessments include but are not limited to: PBMC Assays, Serum Assays, Whole Blood DNA Assays, Whole Blood RNA Assays, Urine Assays, Renal Biopsy Assays, any others provided herein, and combinations thereof.

Procedures

Subjects will undergo a diagnostic renal biopsy at baseline and at Week 38. An additional research biopsy core will be collected at the time of both biopsies.

Unscheduled Visits

Unscheduled (U) Visits. If disease activity increases or other concerns arise between regularly scheduled visits, subjects should be instructed to contact study personnel and may be asked to return to the study site for an “unscheduled” visit. Assessments obtained during an unscheduled (U) visit are outlined in Table 5. Some assessments for an unscheduled visit may be omitted at the discretion of the investigator if not indicated for the purpose of the visit.

Discontinuation (DSC) and Safety Follow-Up Visits (Table 6). Subjects will be followed according to Table 6 in either of the cases such as: VIB4920 or placebo is prematurely discontinued; a randomized subject withdraws consent or otherwise is unable to continue participation in the trial, as described herein; the discontinuation visit (DSC) will be completed within 14 days following premature discontinuation of VIB4920 or placebo, or premature study withdrawal. Subjects will complete one additional safety follow-up visit 12 weeks after DSC (DSC+12). The DSC+12 visit will not be required for subjects who discontinue on or after Week 48. Further care will be provided according to the judgment of the site investigator.

Visit Windows. Study visits should take place within the time limits specified in Table 3. Visit 0 must occur within 28 days of Visit −1. All other scheduled study visits must occur within the time limits specified in Table 3.

TABLE 3
Exemplary Visit Windows

| Visit | Timepoint | Visit Window | |
|---|---|---|---|
| Screening: | Within 28 days prior to Day 0 | | |
| Visit −1 | | | |
| Visit 0 | Day 0 | n/a | |
| Visits 1-2 | Weeks 4 and 8 | +/−5 | days |
| Visits 3 | Week 10 | +/−2 | days |
| Visit 4 | Week 11 | +/−1 | day |
| Visits 5-6 | Weeks 12 and 14 | +/−2 | days |
| Visits 7-11 | Weeks 18, 22, 26, 30, and 34 | +/−5 | days |
| Visit 12 | Week 38 | +/−7 | days |
| | | +21/−7 days for renal | |
| | | biopsy component | |
| Visits 13-14 | Weeks 48 and 60 | +/−14 | days |

Mechanistic Assays

In this study, serial renal biopsy, urine and blood specimens will be collected at the same time points with renal biopsies being limited to two key study visits. The objectives are to analyze the frequencies, phenotypes, and functional profiles of immune cell populations in the target tissue (kidney), urine and blood, and to quantify soluble mediators in serum associated with LN and blockade of CD40:CD40L signaling. These studies will explore immune signatures that correlate with clinical response outcomes, and will help determine what, if any, immunological changes in kidney are paralleled in urine and/or blood by systemic treatment with VIB4920 compared to placebo. Mechanistic studies will address multiple questions, but will be prioritized based on the amount of tissue and blood available.

Mechanistic Hypothesis. B cell germinal center formation, heavy chain class switching and development of protective immune response are dependent on CD40:CD40L interaction. Upregulation of CD40 and CD40L and aberrant expression of CD40L are features of SLE, and B cells are considered to play a significant role in the disease pathogenesis. Therefore, this trial will test the hypothesis that VIB4920 will reduce activated B cells in the LN kidney by interfering with B cell activation through blockade of the CD40:CD40L costimulatory pathway.

Renal Biopsies. This study will collect renal biopsies at baseline and at Week 38 after treatment with VIB4920. Single cell RNAseq on CD45+ cells sorted from frozen renal biopsies using technology developed and performed by the AMP may be used to address the following research questions: Does VIB4920 increase the ratio(s) of naïve to activated B cells, naïve to memory, and/or naïve to ABCs in the kidney? Does VIB4920 reduce the number and activation status of myeloid cells in the kidney? Or introduce a new state? Does VIB4920 decrease the interferon score in myeloid cells in the kidney? Does VIB4920 modulate potentially pathogenic Tfh, Tph and other CD4 T cell subsets, for example CXCR5-PD-1hiCXCR3+IL-10+, and CD8 T cells subsets in the kidney? Clinical pathology renal biopsy slides may be reviewed for structural and immunological changes.

Urine. Urinary lymphocytes and podocytes in LN and other renal diseases have been associated with an inflammatory cell signature and identification of biomarkers of disease activity. Urinary mRNA podocyte damage markers can be used for monitoring risk for progression and response to therapy. Urinary biomarkers of renal response to therapy would be especially useful since it is not feasible for subjects to undergo multiple serial biopsy of the kidney. The immune profile of urine cells is expected to reflect the profile observed in kidney tissue. In addition, the autoreactive profile of urinary B cells may reflect a renal response to therapy more accurately than peripheral blood. Therefore, urine specimens will be collected, and hematopoietic and non-hematopoietic cell populations may be analyzed by single cell flow cytometry and RNA-seq using technology developed and performed by the AMP, and other analytic techniques that become available. Urine proteomics may also be assessed.

Peripheral Blood Cells. Flow cytometry or mass cytometry, single cell RNAseq, ATACseq, and DNA methylation sequencing may be done to analyze the impact of VIB4920 treatment on the frequency and functional status of specific immune cell populations in viable cryopreserved blood leukocytes. Levels of circulating cells will be compared at various time points prior to and following treatment. Additional comparisons will be made between treatment groups to evaluate the effect of treatment on specific cell phenotypes and profiles, and to identify phenotypes and/or profiles that correlate with clinical response outcomes. The following research questions will be addressed: Does VIB4920 reduce the number and frequency of ABCs and plasmablasts in blood? Does VIB4920 decrease the percentage of memory B cells in blood? Does VIB4920 restore the altered DNA methylation and histone code modifications of B cells from subjects with SLE? Does VIB4920 reduce activation of CD14 or CD16 myeloid cells in blood? Does VIB4920 reduce the frequency of Tfh, Tph, or other activated CD4 T cells, for example CXCR5-PD-1hiCXCR3+IL-10+, and/or CD8 T cells in blood? Does VIB4920 reduce expression of interferon-inducible genes in blood leukocytes, secondary to a reduction in autoantibody titers and DNA- or RNA-containing IC?

Serum and Plasma Assays. Levels of soluble immune parameters will be compared at various time points prior to and following treatment. Additionally, levels of soluble immune parameters could be evaluated for correlations with the frequency and activation status of circulating B cell, T cell, and myeloid populations. Finally, comparisons will be made between treatment groups to evaluate the effect of the therapeutic intervention on circulating levels of soluble immune parameters. Subject serum and plasma will be collected and stored for longitudinal analyses using validated platforms to address the following research questions: Does VIB4920 reduce anti-DNA and anti-RNA autoantibody titers? Does VIB4920 modulate levels of CXCL 13, IL-21, and IL-10, and other soluble mediators? Does VIB4920 generate ADA and/or modulate sCD40L levels in plasma?

Whole Blood Assays can comprise RNA and/or DNA assays. In aspects, a whole blood assay comprises an RNA assay. Systemic treatment with biologic medications has been shown to modulate gene expression in autoimmune disease; therefore, whole blood can be used to evaluate changes in the peripheral circulation due to immunomodulation of the disease or the systemic nature of the treatment with VIB4920. Whole blood will be collected from enrolled subjects and may be used to evaluate global changes in gene expression during and after treatment. Gene expression of molecules found to be modulated by treatment in renal tissue, urine and/or blood leukocytes may be investigated in whole blood using quantitative methods. In aspects, a whole blood assay comprises a DNA assay. Genetic differences may, in part, determine response to CD40L blockade with VIB4920. Therefore, DNA will be collected from all consenting subjects to allow HLA typing for specific alleles and single nucleotide polymorphism analysis for genes with reported associations to SLE or drug related effects.

Study Completion

Subject completion. Subjects who are not eligible for randomization will complete the study after the Week 8 study visit. Randomized subjects will complete the study at the Week 60 study visit.

Subject stopping rules and withdrawal criteria. Subjects will be prematurely terminated from the study for the following reasons: (1) Any of the following occur prior to randomization: (a) The subject meets the criteria for worsening proteinuria, as defined herein; (b) The subject meets the criteria for progressive deterioration in renal function, as defined herein; (c) The subject discontinues MMF; (2) The subject elects to withdraw consent from all future study activities, including follow-up; (3) The subject is “lost to follow-up” (i.e., no further follow-up is possible because attempts to reestablish contact with the subject have failed); (4) The subject dies; (5) The Investigator no longer believes participation is in the best interest of the subject.

Subject replacement. Randomized subjects will be replaced if they do not receive any part of any dose of VIB4920 or placebo.

Follow up after premature study withdrawal. If a randomized subject withdraws from the study as described herein and is available, the subject will be asked to complete follow-up as described in Table 6. This will conclude their study participation.

Study stopping rules. Safety data will be review by the DSMB on an ad hoc basis if any of the events listed herein occur. After review of the data, the DSMB will make recommendations regarding study conduct and/or continuation.

Study Definitions are provided in Table 4 below.

TABLE 4
Study Definitions

| Active Lupus Nephritis | Active lupus nephritis will be defined as meeting all of the | |
|---|---|---|
| | following criteria: | |
| | 1. | Diagnosis of Systemic Lupus Erythematosus (1-4) |
| | 2. | Urine protein to creatinine ratio ≥1.5 |
| | 3. | Renal biopsy documentation of ISN/RPS lupus nephritis |
| | | Class III, Class IV, or Class V in combination with Class |
| | | III or IV (5, 6), and |
| | 4. | Modified NIH activity index ≥1 |
| Complete Renal | Complete renal response is defined as meeting all of the | |
| Response | following criteria: | |
| | 1. | Urine protein to creatinine ratio ≤0.75, based on a 24- |
| | | hour urine collection |
| | 2. | Estimated glomerular filtration rate ≥120 ml/min/1.73 m² |
| | | or, if <120 ml/min/1.73 m², then >80% of the estimated |
| | | glomerular filtration rate at baseline |
| | 3. | Prednisone ≤5 mg/day from Week 8, according to the |
| | | prednisone dosing restrictions specified in the protocol |
| Estimated Glomerular | Estimated glomerular filtration rate will be determined by | |
| Filtration Rate | the CKD-EPI formula | |
| Anti-dsDNA | Anti-dsDNA antibodies are defined as any value above the | |
| Antibodies | normal range | |
| Hypocomplementemia | Hypocomplementemia is defined as below the lower limit of | |
| | normal for C3 and/or C4 | |
| Renal Treatment | Renal treatment failure is defined as one or more of the | |
| Failure | following events: | |
| | 1. | Worsening proteinuria, defined as urine protein to |
| | | creatinine ratio ≥1.5 and ≥50% increase in the urine |
| | | protein to creatinine ratio compared to the lowest |
| | | previous value |
| | 2. | Progressive deterioration in renal function, defined as |
| | | serum creatinine ≥1.5 and ≥50% increase in serum |
| | | creatinine compared to the lowest previous value |
| | 3. | Other renal treatment failure events will be evaluated in |
| | | blinded fashion by a renal treatment failure event |
| | | adjudication committee |

| | |
|---|---|
| Anaphylaxis | Anaphylaxis is defined according to that described in: |
| | Sampson H A, Munoz-Furlong A, Campbell R L, Adkinson |
| | N F, Jr., Bock S A, Branum A, et al. Second symposium on |
| | the definition and management of anaphylaxis: summary |
| | report--Second National Institute of Allergy and Infectious |
| | Disease/Food Allergy and Anaphylaxis Network |
| | symposium. J Allergy Clin Immunol. 2006; 117(2): 391-7) |
| | and will be graded according to the National Cancer Institute |
| | Common Terminology Criteria for Adverse Events v5.0 |
| | (Nov. 27, 2017) herein incorporated by reference |
| Renal Biopsy Histology | Renal biopsy histology evaluation will include the following |
| | parameters: ISN/RPS classification, Activity index, |
| | Chronicity index, Vascular lesions including thrombotic |
| | microangiopathy |
| ISN/RPS Lupus | ISN/RPS lupus nephritis will be defined according to the |
| Nephritis | 2018 revisions of the International Society of |
| | Nephrology/Renal Pathology Society classification for lupus |
| | nephritis (Bajema I M, Wilhelmus S, Alpers C E, Bruijn J A, |
| | Colvin R B, Cook H T, et al. Revision of the International |
| | Society of Nephrology/Renal Pathology Society |
| | classification for lupus nephritis: clarification of definitions, |
| | and modified National Institutes of Health activity and |
| | chronicity indices, Kidney international. 2018; 93(4): 789-96; |
| | Weening J J, D'Agati V D, Schwartz M M, Seshan S V, Alpers |
| | C E, Appel G B, et al. The classification of |
| | glomerulonephritis in systemic lupus erythematosus |
| | revisited. Kidney international. 2004; 65(2): 521-30) |
| Lost to Follow-up | No further follow-up is possible because attempts to |
| | reestablish contact with the subject have failed |
| Investigational Agent | VIB4920 or placebo |
| Study Therapy | Study therapy is defined as VIB4920 or placebo, |
| | mycophenolate mofetil, and prednisone |
| Withdrawal from the | Subjects who withdraw or who are withdrawn from the |
| Study | study will be asked to return for safety follow-up visits. |
| Suspected Adverse | Any adverse event for which there is a reasonable possibility |
| Reaction (SAR) | that the investigational agent caused the adverse event. For |
| | the purposes of safety reporting, ‘reasonable possibility’ |
| | means there is evidence to suggest a causal relationship |
| | between the drug and the adverse event. A suspected adverse |
| | reaction implies a lesser degree of certainty about causality |
| | than adverse reaction, which means any adverse event |
| | caused by a drug. |
| Unexpected Adverse | An adverse event or suspected adverse reaction is considered |
| Event | “unexpected” if it is not listed at the specificity, severity or |
| | rate of occurrence that has been observed. |
| | “Unexpected” also refers to adverse events or suspected |
| | adverse reactions as occurring with a class of drugs or as |
| | anticipated from the pharmacological properties of the drug, |
| | but are not specifically mentioned as occurring with the |
| | particular drug under investigation. |
| Serious Adverse Event | An adverse event or suspected adverse reaction is considered |
| (SAE) | “serious” if, in the view of either the investigator or Sponsor |
| | DAIT/NIAID, it results in any of the following outcomes: |
| | (1) Death; (2) A life-threatening event: An AE or SAR is |
| | considered “life-threatening” if, in the view of either the |
| | investigator or Sponsor DAIT/NIAID, its occurrence places |
| | the subject at immediate risk of death. It does not include an |
| | AE or SAR that, had it occurred in a more severe form, |
| | might have caused death; (3) Inpatient hospitalization or |
| | prolongation of existing hospitalization; (4) Persistent or |
| | significant incapacity or substantial disruption of the ability |
| | to conduct normal life functions; (5) Congenital anomaly or |
| | birth defect; (6) Important medical events that may not result |
| | in death, be life threatening, or require hospitalization may |
| | be considered serious when, based upon appropriate medical |
| | judgment, they may jeopardize the subject and may require |
| | medical or surgical intervention to prevent one of the |
| | outcomes listed above. |
| | Elective hospitalizations or hospital admissions for the |
| | purpose of conduct of protocol mandated procedures are not |
| | to be reported as an SAE unless hospitalization is prolonged |
| | due to complications. |
| Adverse Events of | Any occurrence of the following AEs will be considered an |
| Special Interest | Adverse Event of Special Interests (AESI). AESIs must be |
| | reported to the IND Sponsor DAIT/NIAID within 24 hours |
| | of becoming aware of the event, according to the procedure |
| | for SAEs and AESIs described herein: (1) Anaphylaxis, |
| | defined according to Table 7 (8); (2) Grade 3 or greater |
| | infusion reaction; (3) Grade 3 or greater hypersensitivity |
| | reaction; (4) Grade 3 or greater infection; (5) |
| | Thromboembolic event. |
| Safety Sample (SS) | All subjects who receive at least one dose or any part of one |
| | dose of VIB4920 or placebo or the protocol mandated |
| | medications specified herein. The safety analysis will be |
| | based on the actual treatment the subjects receive. |
| Modified Intent to | All randomized subjects who receive at least one dose or any |
| treat (mITT) sample | part of one dose of VIB4920 or placebo. The primary |
| | efficacy analysis will be based on the mITT sample |
| | according to the group to which the subjects are assigned. |
| Per protocol (PP) | All subjects in the mITT sample who meet the following |
| Sample | criteria: (1) No major protocol deviations that impact |
| | efficacy assessments, including use of prohibited |
| | medications and lack of adherence to MMF and prednisone |
| | dosing; (2) Subjects who meet either of the following |
| | criteria: (a) Completed the Week 38 visit assessments and |
| | received at least 7 of the 8 doses of VIB4920 or placebo |
| | infusion, or (b) Discontinued treatment early due to Renal |
| | Treatment Failure and missed no more than one dose of |
| | VIB4920 or placebo prior to discontinuation. The reported |
| | major deviations will be reviewed during a masked data |
| | review after the last subject's primary endpoint visit to |
| | determine which subjects should be excluded from the PP |
| | analysis population. |
| Abbreviations | angiotensin-converting enzyme inhibitor (ACEi), anti-drug |
| | antibodies (ADA), adverse event (AE), Angiotensin receptor |
| | blocker (ARB), area under the curve (AUC), complete blood |
| | count (CBC), Clinical Disease Activity Index (CDAI), |
| | cluster of differentiation 40 ligand (CD40L), Code of |
| | Federal Regulations (CFR), case report form (CRF), contract |
| | research organization (CRO), Disease Activity Score in 28 |
| | Joints Using C-reactive Protein (DAS28-CRP), Data and |
| | Safety Monitoring Board (DSMB), US Food and Drug |
| | Administration (FDA), good clinical practice (GCP), |
| | Investigator brochure (IB), International Conference on |
| | Harmonisation (ICH), institutional review board (IRB), |
| | Immune Tolerance Network (ITN), intravenous (IV), |
| | keyhole limpet hemocyanin (KLH), lupus nephritis (LN), |
| | Medical Dictionary for Regulatory Activities (MedDRA), |
| | modified intent to treat (mITT), mycophenolate mofetil |
| | (MMF), National Cancer Institute Common Terminology |
| | Criteria for Adverse Events (NCI-CTCAE), National |
| | Institute of Allergy and Infectious Diseases (NIAID), |
| | National Institutes of Health (NIH), no-observed-adverse- |
| | events-levels (NOAEL), pharmacodynamics (PD), |
| | pharmacokinetics (PK), per protocol (PP), rheumatoid |
| | arthritis (RA), rheumatoid factor (RF), serious adverse event |
| | (SAE), statistical analysis plan (SAP), subcutaneous (SC), |
| | systemic lupus erythematosus (SLE), Systemic Lupus |
| | Erythematosus Disease Activity Index 2000 (SLEDAI-2K), |
| | Systemic Lupus International Collaborating |
| | Clinics/American College of Rheumatology Damage Index |
| | for Systemic Lupus Erythematosus (SLICC/ACR-DI), safety |
| | sample (SS), half-life (t½), treatment-emergent adverse |
| | event (TEAE), urine protein-to-creatinine ratio (UPCR), |
| | World Health Organization (WHO), Wilcoxan-Mann- |
| | Whitney test (WMW) |

Special Considerations Due to COVID 19

Clinical trial conduct may be impacted by the COVID-19 public health emergency. These impacts may include site closures, travel restrictions, limited availability of site personnel and research space, logistical problems with biospecimens, and SARS-CoV-2 infections in site personnel or trial subjects. These factors may lead to difficulties in completing protocol-specified assessments and procedures, resulting in unavoidable protocol deviations and protocol modifications.

The following principles will guide the conduct of the trial during the COVID-19 public health emergency: Any decisions to begin, continue, or stop enrollment in the study will be made with consideration of the potential impact of the COVID-19 public health emergency on the safety of the subjects. A decision to discontinue the investigational agent or discontinue the subject from the study will be based on the safety and welfare of the subject. This decision will be made by the sponsor DAIT/NIAID, in consultation with the study team and site investigator, and with consideration that VIB4920 may interfere with the ability of the subject's immune system to produce neutralizing antibodies against SARS-CoV-2 and may significantly increase the severity of infection. COVID-19 screening or testing procedures mandated by local health authorities or the health system where the clinical trial is conducted may be performed without the need for a protocol amendment. Urgent changes in the protocol due to SARS-CoV-2 infection or the impact of the public health emergency to protect the safety of subjects may be implemented immediately, with concurrent notification of the IRB. The sponsor DAIT/NIAID will submit revised documents to the health authorities according to current guidance. In certain cases, the subject may not be able to come to the study site, or local institutional policies or other factors may limit access to the research site. The sponsor DAIT/NIAID, in consultation with the study team and site investigator will determine if alternative methods for conducting assessments and procedures are necessary and feasible. Study visit impacts due to the COVID-19 public health emergency will be collected to document and evaluate the potential impact on the safety of the subjects and the outcome of the trial. Failure to conduct required study assessments and procedures or the use of alternative methods to conduct study assessments or procedures will be documented as protocol deviations.

Ethical Considerations and Compliance with Good Clinical Practice

This clinical study will be conducted using good clinical practice (GCP), as delineated in Guidance for Industry: E6 Good Clinical Practice Consolidated Guidance, and according to the criteria specified in this study protocol. Before study initiation, the protocol and the informed consent documents will be reviewed and approved by the IRB. Any amendments to the protocol or to the consent materials will also be approved by the IRB before they are implemented.

TABLE 5
Schedule of Events

| | Week | | | | | | | | | | | | | | | |
|---|---|---|---|---|---|---|---|---|---|---|---|---|---|---|---|---|
| | 0 | 4 | 8 | 10 | 11 | 12 | 14 | 18 | 22 | 26 | 30 | 34 | 38 | 48 | 60 | U¹ |
| Visit | | | | | | | | | | | | | | | | |
| −1 | 0 | 1 | 2 | 3 | 4 | 5 | 6 | 7 | 8 | 9 | 10 | 11 | 12 | 13 | 14 | U |

GENERAL ASSESSMENTS

| | | | | | | | | | | | | | | | | | |
|---|---|---|---|---|---|---|---|---|---|---|---|---|---|---|---|---|---|
| Informed Consent | x | | | | | | | | | | | | | | | | |
| Eligibility criteria | x | | | | | | | | | | | | | | | | |
| Randomization | | | | | x | | | | | | | | | | | | |
| Demographics | x | | | | | | | | | | | | | | | | |
| Medical history | x | | | | | | | | | | | | | | | | |
| Lupus history | x | | | | | | | | | | | | | | | | |
| Comprehensive physical exam | x | | | | x | | | | | | | | | x | | | |
| Limited physical exam | | x | x | x | | | x | x | x | x | x | x | x | | x | x | x |
| Vital signs | x | x | x | x | x | x | x | x | x | x | x | x | x | x | x | x | x |
| Adverse events | | x | x | x | x | x | x | x | x | x | x | x | x | x | x | x | x |
| Concomitant medications | x | x | x | x | x | x | x | x | x | x | x | x | x | x | x | x | x |

DISEASE-SPECIFIC ASSESSMENTS

| | | | | | | | |
|---|---|---|---|---|---|---|---|
| SLEDAI-2K | x | x | x | x | x | x | x |
| SLICC/ACR-DI | x | | x | | | x | |

STUDY MEDICATION

| | | | | | | | | | | | | | | | |
|---|---|---|---|---|---|---|---|---|---|---|---|---|---|---|---|
| MMF | x | x | x | x | x | x | x | x | x | x | x | x | x | x | x |
| Methylprednisolone | x² | | | | | | | | | | | | | | |
| Prednisone² | x | x | x | x | x | x | x | x | x | x | x | x | x | x | x |
| VIB4920 or placebo | | | | x | | x | x | x | x | x | x | x | | | |

CLINICAL LABORATORY ASSESSMENTS

| | | | | | | | | | | | | | | | | |
|---|---|---|---|---|---|---|---|---|---|---|---|---|---|---|---|---|
| Hematology | x | x | x | x | x | x | x | x | x | x | x | x | x | x | x | x |
| Chemistry | x | x | x | x | x | x | x | x | x | x | x | x | x | x | x | x |
| CD19 | x | | | | x | | | x | | x | | | x | x | x | |
| HIV | x | | | | | | | | | | | | | | | |
| Hepatitis B | x | | | | | | | | | | | | | | | |
| Hepatitis C | x | | | | | | | | | | | | | | | |
| Tuberculosis Testing | x | | | | | | | | | | | | | | | |
| Serum pregnancy | x | | | | | | | | | | | | | | | |
| Urine pregnancy | | x | x | x | x | x | x | x | x | x | x | x | x | x | x | x |
| Spot Urine | x | x | x | x | x | x | x | x | x | x | x | x | x | x | x | x |
| Urinalysis | x | | | x | x | | | | | x | | | x | x | x | x |
| 24-Hour Urine | x | | | x | | | | | | x | | | x | x | x | x |
| Serum Immunoglobulins | x | | | | x | | | | | x | | | x | x | x | x |
| Anti-phospholipid Antibodies | x | | | | | | | | | | | | | | | |
| Anti-dsDNA | x | | | | x | | | x | | x | | | x | x | x | x |
| C3, C4 | x | | | | x | | | x | | x | | | x | x | x | x |
| Renal Biopsy Histology | x | | | | | | | | | | | | x | | | |

PROCEDURES

| | | |
|---|---|---|
| Renal Biopsy | x | x |

VIB4920 ASSESSMENTS

| | | | | | | | | | | | | |
|---|---|---|---|---|---|---|---|---|---|---|---|---|
| Plasma PK Assays | x⁴ | x | x⁴ | x | x | x | x | x | x⁴ | x | x | x |
| Urine PK Assays | x | | x | | | | | | x | | | |
| Plasma ADA Assays | x | | x | x | | x | | x | | x | x | |
| Plasma sCD40L Assays | x | | | | | | x | | | x | x | x |

MECHANISTIC ASSESSMENTS⁵

| | | | | | | | | |
|---|---|---|---|---|---|---|---|---|
| PBMC Assays | | x | x | x | x | x | x | x |
| Serum Assays | | x | x | x | x | x | x | x |
| Whole Blood RNA Assays | | x | x | x | x | x | x | x |
| Urine Assays | | x | x | x | x | x | x | x |
| Renal Biopsy Assays | x | | | | | x | | |

¹U = unscheduled visit
²According to disclosure herein
³ Taken daily, beginning at Visit 0, according to disclosure herein
⁴Plasma sample for PK collected pre- and within 10 minutes of post-infusion.
⁵Collect mechanistic blood specimens only when Hgb ≥ 8 g/dL at previous visit.

TABLE 6
Schedule of Events: Discontinuation and Safety Follow-up

| | | |
|---|---|---|
| Week | DSC¹ | DSC + 12² |
| Visit | DSC | DSC + 12 |

GENERAL ASSESSMENTS

| | | |
|---|---|---|
| Comprehensive physical exam | x | |
| Limited physical exam | | |
| Vital signs | x | x |
| Adverse events | | x |
| Concomitant medications | x | x |

DISEASE-SPECIFIC ASSESSMENTS

| | |
|---|---|
| SLEDAI-2K | x |
| SLICC/ACR-DI | x³ |

CLINICAL LABORATORY ASSESSMENTS

| | | |
|---|---|---|
| Hematology | x | x |
| Chemistry | x | x |
| CD19 | x | |
| Urine pregnancy | x | x |
| Spot Urine | x | x |
| Urinalysis | x | |
| 24-Hour Urine | x | |
| Serum Immunoglobulins | x | |
| Anti-dsDNA | x | |
| C3, C4 | x | |

VIB4920 ASSESSMENTS

| | |
|---|---|
| Plasma PK Assays | x |
| Plasma ADA Assays | x |
| Plasma sCD40L Assays | x |

MECHANISTIC ASSESSMENTS

| | |
|---|---|
| PBMC Assays | x |
| Serum Assays | x |
| Whole Blood DNA Assays | x |
| Whole Blood RNA Assays | x |
| Urine Assays | x |

¹DSC = Discontinuation Visit
²DSC + 12 visit will occur 12 weeks following the DSC visit. Not required for subjects who discontinue on or after the Week 48 Visit.
³Collect the SLICC/ACR-DI only if ≥24 weeks have passed since the previous assessment.

TABLE 7
Clinical Criteria for Diagnosing Anaphylaxis
Anaphylaxis is highly likely when any one of the following 3 criteria are fulfilled:

```
1. Acute onset of an illness (minutes to several hours) with involvement of the skin, mucosal tissue, or
  both (e.g., generalized hives, pruritus or flushing, swollen lips-tongue-uvula)
  AND AT LEAST ONE OF THE FOLLOWING
  a. Respiratory compromise (e.g., dyspnea, wheeze-bronchospasm, stridor, reduced PEF, hypoxemia)
  b. Reduced BP or associated symptoms of end-organ dysfunction (e.g., hypotonia [collapse], syncope,
    incontinence)
2. Two or more of the following that occur rapidly after exposure to a likely allergen for that subject
  (minutes to several hours):
  a. Involvement of the skin-mucosal tissue (e.g., generalized hives, itch-flush, swollen lips-tongue-
    uvula)
  b. Respiratory compromise (e.g., dyspnea, wheeze-bronchospasm, stridor, reduced PEF, hypoxemia)
  c. Reduced BP or associated symptoms (e.g., hypotonia [collapse], syncope, incontinence)
  d. Persistent gastrointestinal symptoms (e.g., crampy abdominal pain, vomiting)
  3. Reduced BP after exposure to known allergen for that subject (minutes to several hours):
  a. Infants and children: low systolic BP (age specific) or greater than 30% decrease in systolic BP*
  b. Adults: systolic BP of less than 90 mm Hg or greater than 30% decrease from that person's
    baseline
PEF, Peak expiratory flow; BP, blood pressure.
*Low systolic blood pressure for children is defined as less than 70 mm Hg from 1 month to 1 year, less than (70 mm Hg + [2 × age]) from 1 to 10 years, and less than 90 mm Hg from 11 to 17 years.
Adapted from the Second National Institute of Allergy and Infectious Disease/Food Allergy and Anaphylaxis Network
```

### INCORPORATION BY REFERENCE

This patent application incorporates by reference in their entireties for all purposes the following patent applications: PCT/US2012/059477 and PCT/US2019/052997. All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

We claim:

1. A method for treating lupus nephritis in a subject in need thereof comprising:
administering a Tn3 scaffold comprising a CD40L-specific monomer subunit to the subject,
wherein the Tn3 scaffold binds to CD40L,
wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16,
wherein the Tn3 scaffold comprising the CD40L-specific monomer subunit is administered at a dose of about 1500 mg, and
wherein the Tn3 scaffold is administered once about every 2 weeks for at least 2 doses, and is administered about once a month thereafter.

2. A method for treating lupus nephritis in a subject in need thereof comprising:
administering a Tn3 scaffold comprising a CD40L-specific monomer subunit to the subject,
wherein the Tn3 scaffold binds to CD40L,
wherein the CD40L-specific monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16,
wherein the Tn3 scaffold comprising the CD40L-specific monomer subunit is administered at a dose of about 1500 mg, and
wherein the Tn3 scaffold is administered once about every 2 weeks for at least 3 doses, and is administered about once a month thereafter.

3. A method for treating lupus nephritis in a subject in need thereof comprising:
administering a Tn3 scaffold comprising a CD40L-specific monomer subunit in combination with cyclophosphamide to the subject,
wherein the Tn3 scaffold binds to CD40L,
wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop comprises SEQ ID NO: 16, and
wherein the Tn3 scaffold is administered at a dose of about 1500 mg.

4. The method of claim 3, wherein the cyclophosphamide is administered for at least about eight, ten, twelve or more weeks prior to the administration of the Tn3 scaffold.

5. The method of claim 4, wherein the cyclophosphamide is administered for at least about eight weeks prior to the administration of the Tn3 scaffold.

6. The method of claim 4, wherein the cyclophosphamide is administered for at least about ten weeks prior to the administration of the Tn3 scaffold.

7. The method of claim 4, wherein the cyclophosphamide is administered for at least about twelve weeks prior to the administration of the Tn3 scaffold.

8. The method of any one of claims 2-7, wherein the Tn3 scaffold is administered once about every two, three, four weeks, or about once a month.

9. The method of any one of claims 2-7, wherein the Tn3 scaffold is administered once about every 2 weeks for at least 2 doses, and is administered about once a month thereafter.

10. The method of any one of claims 2-7, wherein the Tn3 scaffold is administered once about every 2 weeks for at least 3 doses, and is administered about once a month thereafter.

11. The method of any of claims 1-10, wherein the Tn3 scaffold is administered intravenously.

12. The method of any one of claims 1-11, wherein the Tn3 scaffold comprises two CD40L-specific monomer subunits connected in tandem.

13. The method of any one of claims 1-12, wherein the Tn3 scaffold:
a. binds CD40L thereby reducing or preventing binding of CD40L to CD40;
b. reduces or eliminates CD40 mediated signaling; or
c. a and b.

14. The method of any one of claims 1-13, wherein at least one CD40L-specific monomer subunit is fused or conjugated to a heterologous moiety selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, and a cytokine.

15. The method of any one of claims 12-14, wherein at least one of the two CD40L-specific monomer subunits is conjugated to PEG or is fused to a human serum albumin (HSA).

16. The method of claim 15, wherein the HSA is a variant HSA comprising the amino acid sequence of SEQ ID NO: 4.

17. The method of any one of claims 1-16, wherein the Tn3 scaffold comprises the sequence of SEQ ID NO: 1.

18. The method of any one of claims 1-17, wherein the Tn3 scaffold is Dazodalibep.

19. The method of any one of claims 1-18, wherein the subject is further administered prednisone.

20. The method of any one of claims 1-19, wherein the subject receives one or more standard of care therapies prior to the administration of the Tn3 scaffold.

21. The method of claim 20, wherein the standard of care therapy is continued subsequent to the administration of the Tn3 scaffold.

22. The method of claim 20, wherein the standard of care therapy is cyclophosphamide.

23. The method of claim 20, wherein the standard of care therapy is prednisone.

24. The method of any of the preceding claims, wherein the beta A strand comprises SEQ ID NO: 5, SEQ ID NO: 23, or SEQ ID NO: 24, wherein the beta B strand comprises SEQ ID NO: 6, wherein the beta C strand comprises SEQ ID NO: 17, wherein the beta D strand comprises SEQ ID NO: 18, wherein the beta E strand comprises SEQ ID NO: 19, wherein the beta F strand comprises SEQ ID NO: 20, and wherein the beta G strand comprises SEQ ID NO: 21.

25. The method of claim 24, wherein the beta A strand consists of SEQ ID NO: 5.

26. The method of claim 24, wherein the beta A strand consists of SEQ ID NO: 23.

27. The method of claim 24, wherein the beta A strand consists of SEQ ID NO: 24.

28. The method of claim 24, wherein the beta B strand consists of SEQ ID NO: 6.

29. The method of claim 24, wherein the beta C strand consists of SEQ ID NO: 17.

30. The method of claim 24, wherein the beta D strand consists of SEQ ID NO: 18.

31. The method of claim 24, wherein the beta E strand consists of SEQ ID NO: 19.

32. The method of claim 24, wherein the beta F strand consists of SEQ ID NO: 20.

33. The method of claim 24, wherein the beta G strand consists of SEQ ID NO: 21.
